SEQUENCE LISTING (4 sequences)

SEQ ID NO: 1 - DNA, 1185 bp, Homo sapiens
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaataactgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccagaatcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 2 - DNA, 7040 bp, Homo sapiens
agttttgtttttttttttttcggcggagatcctcgttggggctgggaaactcctgcaaaactcgagaccaggaagccagcccgcaccccaacccccaccaaagccacctactcttcttctgtgggaggccagtccacatccgctctcacccgagagagatattcagctggatccaaagtgactgatgaagggaaggaaatcatgtcaagcgaagccttgaaaaagctgccctgagacggtgtcccgccgaaagaatgttggctcaattaagaaacatcagggagataaattcaacccagtgtgtctaaaaatgactacaaaacgaagtttgtttgtgcggttggtaccatgtcgctgtctacgaggggaagaggagactgtcactactcttgattattctcattgcagcttagaacaagttccgaaagagatttttacttttgaaaaaaccttggaggaactctatttagatgctaatcagattgaagagcttccaaagcaactttttaactgtcagtctttacacaaactgagtttgccagacaatgatttaacaacgttaccagcatccattgcaaaccttattaatctcagggaactggatgtcagcaagaatggaatacaggagtttccagaaaatataaaaaattgtaaagttttgacaattgtggaggccagtgtaaaccctatttccaagctccctgatggattttctcagctgttaaacctaacccagttgtatctgaagatgcttttcttgagttcttgccagcaaattttggcagattaactaaactccaaatattagagcttagagaaaaccagttaaaaatgttgcctaaaactatgaatagactgacccagctggaaagactggatttgggaagtaacgaattcacggaagtgcctgaagtacttgagcaactaagtggattgaaagagttttggatggatgctaatagactgacttttattccagggtttattggtagtttgaaacagctcacatatttggatgtttctaaaaataatattgaaatggttgaagaaggaatttcaacatgtgaaaaccttcaagacctcctattatcaagcaattcacttcagcagcttcctgagactattggttcgttgaagaatataacaacgcttaaaatagatgaaaaccagttaatgtatctgccagactctataggagggttaatatcagtagaagaactggattgtagtttcaatgaagttgaagctttgccttcatctattgggcagcttactaacttaagaacttttgctgctgatcataattacttacagcagttgcccccagagattggaagctggaaaaatataactgtgctgtttctccattccaataaacttgagacacttccagaggaaatgggtgatatgcaaaaattaaaagtcattaatttaagtgataatagattaaagaatttaccctttagctttacaaagctacagcaattgacagctatgtggctctcagataatcagtccaaacccctgatacctcttcaaaaagaaactgattcagagacccagaaaatggtgcttaccaactacatgttccctcaacagccaaggactgaggatgttatgtttatatcagataatgaaagttttaacccttcattgtgggaggaacagaggaaacagcgggctcaagttgcatttgaatgtgatgaagacaaagatgaaagggaggcacctcccagggagggaaatttaaaaagatatccaacaccatacccagatgagcttaagaatatggtcaaaactgttcaaaccattgtacatagattaaaagatgaagagaccaatgaagactcaggaagagatttgaaaccacatgaagatcaacaagatataaataaagatgtgggtgtgaagacctcagaaagtactactacagtaaaaagcaaagttgatgaaagagaaaaatatatgataggaaactctgtacagaagatcagtgaacctgaagctgagattagtcctgggagtttaccagtgactgcaaatatgaaagcctctgagaacttgaagcatattgttaaccatgatgatgtttttgaggaatctgaagaactttcttctgatgaagagatgaaaatggcggagatgcgaccaccattaattgaaacctctattaaccagccaaaagtcgtagcacttagtaataacaaaaaagatgatacaaaggaaacagattctttatcagatgaagttacacacaatagcaatcagaataacagcaattgttcttctccatctcggatgtctgattcagtttctcttaatactgatagtagtcaagacacctcactctgctctccagtgaaacaaactcatattgatattaattccaaaatcaggcaagaagatgaaaattttaacagccttttacaaaatggagatattttaaacagttcaacagaggaaaagttcaaagctcatgataaaaaagattttaacttacctgaatatgatttgaatgttgaagagcgattagttctaattgagaaaagtgttgactcaacagccacagctgatgacactcacaaattagatcatatcaatatgaatcttaataaacttataactaatgatacatttcaaccagagatcatggaaagatcaaaaacacaggatattgtgcttggaacaagctttttaagcattaattctaaagaggaaactgagcacttggaaaatggaaacaagtatcctaatttggaatccgtaaataaggtaaatggacattctgaggaaacttcccagtctcctaatggactgaaccacatgacagtgattgttctgttgacttaggtatttccaaaagcactgaagatctctcccctcagaaaagtggtccagttggatctgttgtgaaatctcatagcataactaatatggagattggagggctaaaaatctatgatattcttagtgataatggacctcagcagccaagtacaaccgttaaaatcacatctgctgttgatggaaaaaatatagtcaggagcaagtctgccacactgttgtatgatcaaccattgcaggtatttactggttcttcctcatcttctgatttaatatcaggaacaaaggcaattttcaagtttgattcaaatcataatcccgaagagccaaatataataagaggccccacaagtggcccacaatctgcacctcaaatatatggtcctccacagtataatatccaatacagtagcagtgctgcagtcaaagacactttgtggcactccaaacaaaatccccaaatagaccatgccagttttcctcctcagctccttcctagatcagagagcacagaaaatcaaagttatgctaaacattctgccaatatgaatttctctaatcataacaatgttcgagctaatactgcataccatttacatcagagacttggcccagcaagacatggggaaatgtgggccatctcaccaaacgaccgacttattcctgcagtaactcgaagtacaatccagcgacaaagtagtgtgtcctccacagcctctgtaaatcttggtgatccaggctctacaaggcgggctcagattcctgaaggagattatttatcatacagagagttccactcagcgggaagaactcctccaatgatgccaggatcacagagacccctttctgcacgaacatacagcatagatggtccaaatgcatcaagacctcagagtgctcgaccctctattaatgaaataccagagagaactatgtcagttagtgatttcaattattcacggactagtccttcaaaaagaccaaatgcaagggttggttctgagcattctttattagatcctccaggaaaaagtaaagttcctcgtgactggagagaacaagtacttcgacatattgaagccaaaaagttagaaaagaagcatccccagacatccagttcaggagatccttgtcaagatggtatattcatttcaggacagcagaactactcatcagccacacttagtcacaaagatgttcctccagacagcttgatgaaaatgcctttgagtaatggacagatgggccagcctctcaggcctcaggcaaattatagtcaaatacatcacccccctcaggcatctgtggcaaggcatccctctagagaacaactaattgattacttgatgctgaaagtggcccaccagcctccatatacacagccccattgttctcctagacaaggccatgaactggcaaaacaagagattcgagtgagggttgaaaaggatccagaacttggatttagcatatcaggtggtgtcgggggtagaggaaacccattcagacctgatgatgatggtatatttgtaacaagggtacaacctgaaggaccagcatcaaaattactgcagccaggtgataaaattattcaggctaatggctacagttttataaatattgaacatggacaagcagtgtccttgctaaaaactttccagaatacagttgaactcatcattgtacgagaagtttcctcataagcactgtggacaaaaaaagcggggaagacagcaagatttattggaagatacttacaggggaaattaatattttgactatttttatatataaagaagaactcaaaaaattatgttcaaatttgtacattaatgaaataatggaacttgtggttagagggaaagaaccactgtacagaatataaaggagactgttgaattcataccatataaaacttgttaggtttttaaacatagcaatcaaggctacaaaaacaaacctgtgttgtttttgtatagattgtaggtttatttttggatttcatatacatgactgaactgtgtgcaaggcaatagttagccttgattttagcccagagacagatggcagagctatctctctcatagcttttatgcccttatttttattcaactggtattaatgtttttctcctgaaactactttttttgatgtgggcaagagatttgaagtgttggcttttgctatgtgcatattgaattgaagagtgagtaggtgaaggtggtgctggtgggttcactttccaaggccagactaaaacagttattttctataaaaatctggaagcaaagaatggggatggggagagctacgtggtagtatgtttttattaggagaataatgcaataaaatatgtaatgtcttttttataaagcaaaaaagacaataattgcatttatgagctcggcaggatctgttcttgtcatagccattgactatacatttgctactggtgattcagtttttaattttttagtcacaggaaatttttaactctactgtagatgcatgtccatgcattttctgtgttatggaaatccactgatttttttttttttttcaaatggtggtacttgcaatctgttttataattagtgctccatttaaatctaatttataatttttattttaagcagcaaatgaaacaaaaatggccagttttaagattgtgttgcctgtaacacaaaatgttacgaaggtttaggaaagcctctttgatttttgtttggccttgcattgccttggtaaagtaaaaggaaacagtacacttggagctaggaaaccaaagcaagctttgtgaaactggcacagtgatagagaattgctgtggagagttatagagcaaagggatgggtccttgaggcctgccagtgtgtaaaggtgttcaaataaagggctgtttctacaggtaacattaaatgtgaactcaacacttccagagtctttaaagggtttctatgtgtatcagtgtaatagtgttttaccaccaactgcctttctttgttcctagttactgtaacaaatatttgatgatagaggtttattaattttgtttatccagaccattaattttatttgtttttgttctatgtaatcaaataaaatttgagtaacatgtaatggtaaggattaatgcatggttatttggaccagaaaaaagtgccatagaagaccaataactgtttagttgaggctagtctggaacctttcattagagcaatatttggttattgcacttcatttttatttactaagaaatgcaatttgggaatttttaatctgttatgctttgtttatcaaccttgattttaattaagacttttataagactagcttaaaacaccaaccaacattatttttgcaaaagtgagttggactcactttccattcttgctagtcagagtaagtaggcagcacttttaaaaatatgtgaactcaaatattgcacttctttcaagatgttatcaattggttattgtactgtatagttttaataattttgattgaaaccctttaacaactctttgtaaattttaactcattttagttgattttcagtactatttacataggaattgatttttatggatatagtagaagaaatgtgctgtattttgataaaattcacttattgtatgtgtgttgtaatctaaaaaaaaaaagaatgacaaacagcttctttaagacaagtctcggtgttccctttattcttagtttgtttttaaatattaattttggcattctaaaatagctaacatttcttttattgatttcagattttcacaggcacattctacttttaatcagaaatatatttaataagtataattgtgaagttttcaactactttaccttgaaccacatataccaattataattttggaaaaggaattaagcctcacggaacaatggatcttcagcaaaccttaacttcattgtctgcacattacattgaagtattataaatgcaacagatgttatatgcactggcattttatcctactctagttagttaaaattttatagtattcttgcaacacataaagttgcgtaagaaactttaccaagaggagtattatagccaagttttctttgaaagtattggaaaactaaaattaaatgacaaggactttgaattagaattttgctgtaataaagtttcaaaatttgaataaaataattaaattttttgagga

SEQ ID NO: 3 - DNA, 2583 bp, Homo sapiens
ctacctcccagggaatgaaagctactggttgattttaaagtgcctgggcctcacaggtttggagatgtcccagaataaggcacaatgtcaatagcaggagttgctgctcaggagatcagagtcccattaaaaactggatttctacataatggccgagccatggggaatatgaggaagacctactggagcagtcgcagtgagtttaaaaagtaagtagaggatgtaatgctgctgtaatctggataaatatgtgacactaaaatgggagaggctgtgattgctcttcgcttatgaccaaagtagcttcctctcctttcagcaactttttaaatattgacccgataaccatggcctacagtctgaactcttctgctcaggagcgcctaataccacttggtatgtattctgaaaatctgatcacagtaagcatttgagaagaacagtctggattcgggttagcttgtcctccagcattattttttaaatgaggaaacctgaactatttccaacaacagcctgacccctagtggcaacagattcagaagataactgtgtttttctcaagctattgtactcgactgccttcattctgagtcactgattgctaagtaggactgttcatggacgtgggatcttctaaaatcaagaattagttctcattccagctctgatgcatactttacttcatgaaaccttaggcgagatttcccacctttcttactagtatcgaatgcatgtttgacagtaatagatgaaaatagtataaatgttcctcaaaacttaaaaaatagtatttttaatgtgaatattctgttccttggatctttgtcaagagctgtgtgtgaactgaacacattgcaggcaagtccattcactcacaatattatgatgggccagcaataaggactttgtcttatctcattggtaccctacgtgcctagtatggtcgcatgtcttaaatggcaaggctggtacagtatggtattcatgtaaattatatgctattcatcttccgcgaattttacacacgtcacaaaacttgcctgtgatgtgtgggtgtgcgctgtgcacatgtccaagggagatagaggagatagtttgttctttgaaccacaccatgtgcgttaagaatcttctgctctctaattacacctgtggtggttgcatgggtgttctcggggtgacagcagtcaagtgtttcactcaggaagaaagctgtggaagcataggtagctggggtgctctctccctcacacaggtggagagaggattgttgatcttttattaatatctctcgttcattccagggcatgcttccaaatctgctccgatgaatggccactgctttgcagaaaatggtccatctcaaaagtccagcttgccccctcttcttattcccccaagtgaaaacttgggaccacatgaagaggatcaagttgtatgtggttttaagaaactcacagtgaatggggtttgtgcttccacccctccactgacacccataaaaaactccccttcccttttcccctgtgcccctctttgtgaacggggttctaggcctcttccaccgttgccaatctctgaagccctctctctggatgacacagactgtgaggtggaattcctaactagctcagatacagacttccttttagaagactctacactttctgatttcaaatatgatgttcctggcaggcgaagcttccgtgggtgtggacaaatcaactatgcatattttgataccccagctgtttctgcagcagatctcagctatgtgtctgaccaaaatggaggtgtcccagatccaaatcctcctccacctcagacccaccgaagattaagaaggtctcattcgggaccagctggctcctttaacaagccagccataaggatatccaactgttgtatacacagagcttctcctaactccgatgaagacaaacctgaggttccccccagagttcccatacctcctagaccagtaaagccagattatagaagatggtcagcagaagttacttcgagcacctatagtgatgaagacaggcctcccaaagtaccgccaagagaacctttgtcaccgagtaactcgcgcacaccgagtcccaaaagccttccgtcttacctcaatggggtcatgcccccgacacagagctttgcccctgatcccaagtatgtcagcagcaaagcactgcaaagacagaacagcgaaggatctgccagtaaggttccttgcattctgcccattattgaaaatgggaagaaggttagttcaacacattattacctactacctgaacgaccaccatacctggacaaatatgaaaaattttttagggaagcagaagaaacaaatggaggcgcccaaatccagccattacctgctgactgcggtatatcttcagccacagaaaagccagactcaaaaacaaaaatggatctgggtggccacgtgaagcgtaaacatttatcctatgtggtttctccttagaccttggggtcatggttcagcagaggttacataggagcaaatggttctcaattttccagtttgattgaagtgcagagaaaaatccctta

SEQ ID NO: 4 - DNA, 5709 bp, Homo sapiens
ggctgtcctctcaccgtcctcaccccgcgaggcccggcccgctcctccgtcgtggatttcgcggcgatccccccggcagctctttgcaaagctgcttgaaacttctcccaaactcggcatggatacgactgcggcggcggcgctgcctgcttttgtggcgctcttgctcctctctccttggcctctcctgggatcggcccaaggccagttctccgcaggtggctgtacttttgatgatggtccaggggcctgtgattaccaccaggatctgtatgatgactttgaatgggtgcatgttagtgctcaagagcctcattatctaccacccgagatgccccaaggttcctatatgatagtggactcttcagatcacgaccctggagaaaaagccagacttcagctgcctacaatgaaggagaacgacactcactgcattgatttcagttacctattatatagccagaaaggactgaatcctggcactttgaacatattagttagggtgaataaaggacctcttgccaatccaatttggaatgtgactggattcacgggtagagattggcttcgggctgagctagcagtgagcaccttttggcccaatgaatatcaggtaatatttgaagctgaagtctcaggagggagaagtggttatattgccattgatgacatccaagtactgagttatccttgtgataaatctcctcatttcctccgtctaggggatgtagaggtgaatgcagggcaaaacgctacatttcagtgcattgccacagggagagatgctgtgcataacaagttatggctccagagacgaaatggagaagatataccagtagcccagactaagaacatcaatcatagaaggtttgccgcttccttcagattgcaagaagtgacaaaaactgaccaggatttgtatcgctgtgtaactcagtcagaacgaggttccggtgtgtccaattttgctcaacttattgtgagagaaccgccaagacccattgctcctcctcagcttcttggtgttgggcctacatatttgctgatccaactaaatgccaactcgatcattggcgatggtcctatcatcctgaaagaagtagagtaccgaatgacatcaggatcctggacagaaacccatgcagtcaatgctccaacttacaaattatggcatttagatccagataccgaatatgagatccgagttctacttacaagacctggtgaaggtggaacggggctcccaggacctccactaatcaccagaacaaaatgtgcagaacctatgagaaccccaaagacattaaagattgctgaaatacaggcaagacggattgctgtggactgggaatccttgggttacaacattacgcgttgccacacttttaatgtcactatctgctaccattacttccgtggtcacaacgagagcaaggcagactgtttggacatggaccccaaagcccctcagcatgttgtgaaccatctgccaccttatacaaatgtcagcctcaagatgatcctaaccaatccagagggaaggaaggagagtgaagagacaattattcaaactgatgaagatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggaatacccggcaggagatgactcacatggtgaatgcaatggatcgaagttatgctgatcagagcactctgcatgcagaagatcctctttccatcaccttcatggaccaacataactttagtccaagatatgagaaccacagtgctacagcagagtccagtcgccttctagacgtacctcgctacctctgtgaggggacggaatccccttaccagacaggacagctgcatccagccatcagggtagctgatttactgcagcacattaatctcatgaagacatcagacagctatgggttcaaagaggaatatgagagcttttttgaaggacagtcagcatcttgggatgtagctaaaaaagatcaaaatagagcaaaaaaccgatatggaaacattatagcatatgatcactccagagtgattttgcaacccgtagaggatgatccttcctcagattatattaatgccaactatattgatatttggctgtacagggatggctaccagagaccaagtcattacattgcaacccaaggtcccgttcatgaaacagtgtatgatttctggaggatgatttggcaagaacaatctgcttgcattgtgatggttacaaatttagttgaggttggccgggttaaatgctataaatattggcctgatgatactgaagtttatggtgacttcaaagtaacgtgtgtagaaatggaaccacttgctgaatatgtagttaggacattcaccctggaaaggagggggtacaatgaaatccgtgaagttaaacagttccatttcacgggctggcctgaccatggagtgccctaccatgctacagggctgctttcctttatccggcgagtcaagttatcaaaccctcccagtgctggccccatcgttgtacattgcagtgctggtgctggacgaactggctgctacattgtgattgacatcatgctagacatggctgaaagagagggtgttgttgatatttacaattgtgtcaaagccttaagatctcggcgtattaatatggtccagacagaggaacagtacatttttattcatgatgccattttagaagcctgcttatgtggagaaactgccatacctgtctgtgaatttaaagctgcatattttgatatgattagaatagactcccagactaactcttcacatctcaaggatgaatttcagactctgaattcagtcacccctcgactacaagctgaagactgcagtatagcgtgcctgccaaggaaccatgacaagaaccgtttcatggacatgctgccacctgacagatgtctgccttttttaattacaattgatggggagagcagtaactacatcaatgctgctcttatggacagctacaggcaaccagctgctttcatcgtcacacaataccctctgccaaacactgtaaaagacttctggagattagtgtatgattatggctgtacctccattgtgatgttaaacgaagtcgacttgtcccagggctgccctcagtactggccagaggaagggatgctacgatatggccccatccaagtggaatgtatgtcttgttcaatggactgtgatgtgatcaaccggatttttaggatatgcaatctaacaagaccacaggaaggttatctgatggtgcaacagtttcagtacctaggatgggcttctcatcgagaagtgcctggatccaaaaggtcattcttgaaactgatacttcaggtggaaaagtggcaggaggaatgcgaggaaggggaaggccggacgattatccactgcctaaatggtggcgggcgaagtggcatgttctgtgctataggcatcgttgttgaaatggtgaaacggcaaaatgttgtcgatgttttccatgcagtaaagacactgaggaacagcaagccaaacatggtggaagccccggagcaataccgtttctgctatgatgtagctttggagtacctggaatcatcttagttgggtgagactctttaaagtgcatccatgaagaaacctgtccatctattgagccagcagctgttgtacctgttacacttgtgcagaaagattttaatgtggggggtgggagacttttacatttgagaggtaaaagtattttttttatgaagttgtgtatcttaataaaaaggactgaattagtttttattactatattaaagcatcaacatttcatgccacataaattatatttaataagaaccagattgaaatgagaacgtattggtgtttgtacagtgaacatgccaccttttttctcatggtttcagtagagcagctaccacatgttgcatgagttcatactttctacgtggcatttttctccctttctaaaatgaaagctgatgaatcttaaaaggaagaagaaaagaaaagctgtgcaaattcatagtaaagttcgttttttatatgtttccagtgtagcagatctctatataaatatataaatatatataactggcttattttcttttaatgtgcaatgatggctggatcatttaaagttctttttagaaaataacataagccaaagactcaagtgtaaatatgtctatatggagaaagcacattatatttattggttacttacattccttttttgatggctaaaatactaccaccacacaatcatcttttttttcctgaagaaagctttttctttagctaaaatcaattgtaaacgatttttgtagattattttttgtatgttttagtgtaagtagaagataaactttttattcataaaccaggaagcaatgttctttatagtgattctcttgtgtacatgcttgtgaattaaatttgtgtaaaatcccttggcaattgggtcttttaatataggaccaaattaaaacattttgctgaatatgtatagtttttcacaatttcattaggtaaataatggtttggtgatcatacatgagaaatgtacacattaaaaggccttgctgacaacttgcacaatgttgaacatagcctttaagcatcatttaaattttaaaggaatggagtttttcagcctgtggcccagcactggtcaagaaaacaagatggcaacatatatgctttcagggtcaaatttgagcaaactgtaaactgtcagggtgataaaatgtttctcttgatgtttacatgcacaagctttgcgttctgactataaaaagtgtgaacaaatcaatgccagattcctgttttgcgcattgtcatgg

(19) United States
(12) Patent Application Publication
(10) Pub. No.: US 2020/0093824 A1
(43) Pub. Date: Mar. 26, 2020

(54) ERBB3 Activators in Hearing Restoration

(71) Applicant: University of Rochester, Rochester, NY (US)

(72) Inventors: Patricia M. White, Rochester, NY (US); Jingyuan Zhang, Rochester, NY (US); Albert Edge, Boston, MA (US)

(73) Assignee: University of Rochester, Rochester, NY (US)

(21) Appl. No.: 16/561,475

(22) Filed: Sep. 5, 2019

Related U.S. Application Data
(60) Provisional application No. 62/727,319, filed on Sep. 5, 2018.

(51) Int. Cl. A61K 31/506; C12N 5/0793; A61K 9/00; A61K 31/713; C12N 15/113; A61P 27/16

(52) U.S. Cl. CPC A61K 31/506; C12N 5/062; A61K 9/0019; A61K 9/0046; C12N 2320/32; C12N 15/113; A61P 27/16; C12N 2501/999; C12N 2310/14; A61K 31/713

(57) ABSTRACT

This invention relates to uses of activators of ErbB3/HER3 in expanding inner ear cells and restoring hearing loss.

### CROSS REFERENCE TO RELATED APPLICATION

This application claims priority to U.S. Provisional Application No. 62/727,319 filed on Sep. 5, 2018. The content of the application is incorporated herein by reference in its entirety.

### GOVERNMENT INTERESTS

This invention was made with government support under DC014261 and DC014089 awarded by National Institutes of Health. The government has certain rights in the invention.

### FIELD OF THE INVENTION

This invention relates to uses of activators of ErbB3/HER3 in expanding inner ear cells and restoring hearing loss.

### BACKGROUND OF THE INVENTION

Hearing loss affects about 12% of individuals over the age of twelve, or around 30 million Americans (NIDCD 2010). The likelihood of having bilateral hearing loss doubles each decade after the age of fifty (Bainbridge and Wallhagen 2014), to the point that over 60% of people aged 70 or older have hearing loss (Lin, Thorpe et al. 2011). Outer hair cell (OHC) loss is a significant factor in many kinds of hearing loss (Crowe, Guild et al. 1934, McGill and Schuknecht 1976), largely because these specialized acoustic amplifying cells do not regenerate their numbers if they die (Chardin and Romand 1995). There is a need for therapeutic and methods for expanding OHC and other inner ear cells thereby restoring hearing loss.

### SUMMARY OF INVENTION

This invention relates to methods for expanding inner ear cells and restoring hearing loss. Accordingly, in one aspect, the invention provides a method of expanding a population of inner ear cells. The method comprises contacting the cells with an effective amount of an inhibitor of a negative ERBB3 regulator or a pharmaceutically acceptable salt of the inhibitor. Examples of the negative ERBB3 regulator include Proliferation-Associated 2G4 (PA2G4) (also known as ERBB3 binding protein 1, EBP1), Erbb2 interacting protein (ERBIN), ERBB receptor feedback inhibitor 1 (ERRFI1) and Protein Tyrosine Phosphatase, Receptor Type K (PTPRK). Examples of a PA2G4 inhibitor include WS3, WS6, or a derivative thereof. Additional examples include a nucleic acid, such as an antisense nucleic acid or a siRNA molecule, which targets PA2G4, ERBIN, ERRFI1, or PTPRK RNA. The inner ear cells can be Myo7⁺, Atoh1⁺ OCM⁺, Prestin⁺, or VGLUT3⁺. Examples can be one or more selected from the group consisting of inner hair cells, outer hair cells, vestibular hair cells, cochlear cells and vestibular supporting cells. To practice the method, the inner ear cells can be in vitro or in vivo. In one embodiment, the population of inner ear cells are in a cochlear tissue. The cochlear tissue can be in vitro or in vivo in a subject. The subject can be a mammal, such as a human.

In a second aspect, the invention provides a method of treating hearing loss in a subject in need thereof. The method includes applying to the inner ear or the organ of Corti of the subject an effective amount of an inhibitor mentioned above. The inhibitor can be administered into the scala tympani or the scala media. The inhibitor can be administered by any suitable means known in the art, including intratympanic administration and intracochlear administration using microneedle/syringe, nanoparticles, cell-penetrating peptides, magnetic force, gel, ear cube, viral vectors, or apical injections. The inhibitor can be in a sponge, a gel, a biopolymer, a tubing, or a pump. Examples of the PA2G4 inhibitor include WS3, WS6, a derivative thereof, and a nucleic acid that targets PA2G4 RNA (such as an antisense nucleic acid or a siRNA molecule). In some embodiments, the PA2G4 inhibitor can be injected at 0.005-60 ng/injection, such as 0.01-30 ng/injection.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, 1D, 1E, and 1F are a set of diagrams and photographs showing viral constitutively active ERBB2 gene (CA-ERBB2) transduction drives ERBB2 phosphorylation and downstream signaling. FIG. 1A. Schematic of CA-ErbB2 receptors showing the mechanism of dimerization. Asterisk indicates approximate region of mutation. FIG. 1B. Mouse brain fibrocytes were separately infected with 3 viral constructs, GFP (1), I-ErbB2 (2) and CA-ErbB2 (3) for 24 hours. Their protein extracts were analyzed in western blot with an antibody against ERBB2. FIG. 1C. Same extracts, probed with an antibody against phosphorylated ERBB2. FIG. 1D. Same extracts, probed with an antibody against the phosphorylated PI3K regulatory subunit. FIG. 1E. Same extracts, probed with an antibody against β-ACTIN. All four panels were processed concurrently. FIG. 1F. Semi-quantitation of western blots (ImageJ). The y axis shows arbitrary units.

FIGS. 2A, 2B, 2C, 2D, 2E, 2F, and 2G are a set of photographs showing viral CA-ERBB2 transduction in P2 cochlear cultures drives JAG1+ supporting cell (SC) proliferation in a non-cell autonomous, which does not co-localize with SOX2. P2 cochlear middle turns were infected in vitro by each of the 3 viruses: GFP (FIGS. 2A and 2B), I-ErbB2 (FIGS. 2C and 2D), and CA-ErbB2 (FIGS. 2E and 2F). Anti-JAG1 (FIGS. 2A, 2C, and 2E, red) and anti-SOX2 (FIGS. 2B, 2D, and 2F, cyan) are used to identify SCs. Staining for GFP (green) and EdU (white) reveal infected and proliferating cells, respectively. A schematic for quantifying fields without bias is shown (FIG. 2G). Similar numbers of JAG1+/EdU+ cells were seen in GFP-infected and I-ErbB2 infected cultures (39.5±9.2 EdU+ cells/mm vs 37.6±9.9 EdU+ cells/mm, p=0.89, two-tailed t-test, n=6-8 organs per condition), but significantly more were seen after CA-ERBB2 infection (72.9±11.2 EdU+ cells/mm, p=0.04, two-tailed t-test, n=10 organs for CA-ERBB2 and 8 organs for GFP; ANOVA for all three conditions, p=0.04). However, few SOX2+/EdU+ nuclei are observed (cf. FIGS. 2E and 2F). Scale bar: 200 microns.

FIGS. 3A, 3A′, 3A″, 3B, 3B′, 3B″, 3C, 3C′, 3C″, 3D, 3D′, and 3D″ are a set of photographs showing Sox2 downregulation in cochlear SCs during proliferation in vitro. Cochleae derived from Sox2-Creert/ROSA-floxed Tomato pups were first cultured with 6-hydroxytamoxifen, to genetically label SOX2+ SCs with TOMATO protein, and then infected with either GFP virus (FIGS. 3A, 3A′, and 3A″) or CA-ErbB2 virus (FIGS. 3B, 3B′, 3B″, 3C, 3C′, 3C″, 3D, 3D′, and 3D″). Cultures were allowed to incubate with EdU for 24 (FIGS. 3A, 3A′, 3A″, 3B, 3B′, 3B″, 3C, 3C′, and 3C″) or 32 hours (FIGS. 3D, 3D′, and 3D″) before fixation. Various combinations of staining are displayed, with EdU (white), TOMATO (designating the Sox2 lineage, red), GFP (designating viral infection, green), and SOX2 protein (cyan) as indicated. Pink arrows indicate infected cells; yellow arrows indicate EdU+ cells. Projections from confocal stacks with side views are presented to place EdU+ nuclei in the context of TOM and SOX2 (FIGS. 3C, 3C′, 3C″, 3D, 3D′, and 3D″). 79.4%±4.6% of EdU+/TOM+ nuclei were negative for SOX2 protein. (FIGS. 3C, 3C′, 3C″) shows projections from the area indicated with an arrow in (FIGS. 3B, 3B′, and 3B″); (FIGS. 3D, 3D′, and 3D″) shows an additional image from a separate experiment fixed at 32 hours. Scale bar: 50 microns.

FIGS. 4A, 4B, 4C, 4D, 4D′, 4E, 4E′, 4F, 4G, 4G′, 4G″, 4H, 4H′, 4H″, 4I, 4I′, and 4I″ are a set of diagrams and photographs showing activation of CA-ERBB2 in cochlear SCs at neonatal stages drives SOX2 downregulation in vivo. FIG. 4A. Western analysis of ErbB2, phosphor-ErbB2, β-actin, and phosphor-PI3K protein levels (clockwise from upper left, L=MW ladder.) The lysates were obtained from cultured brain fibrocytes from ROSA-rtTA+/CA− ErbB2+ mice (lane a) and ROSA-rtTA mice (lane b). All four panels were processed concurrently from the same lysates 24 hours after dox addition. FIG. 4B. Western analysis of CA-ErbB2 protein induction in CA-ErbB2/ROSA-rtTA derived fibrocytes. Samples were harvested 2, 4, 6 and 8 hours after dox addition. FIG. 4C. Example breeding scheme used to generate mice for these experiments. Fgfr3-iCre is shown; Sox2-CreERT mice were similarly bred. A red X is placed over symbols for genes if the protein cannot be expressed in that genetic combination. Only mice harboring all three modifications can express CA-ERBB2. Note that the ROSA-flox-rtTA modification includes an IRES-GFP (not shown), which enables lineage tracing of cells where that locus is recombined after CRE activation. FIGS. 4D, 4D, and 4E. GFP produced along with TA protein from the ROSA locus (FIG. 4D, 4D′, and 4E, green) co-localizes with p-ERBB2 in mice harboring both Sox2-CreERT, CA-ErbB2, and ROSA-flox-rtTA genes (FIG. 4D′, red) but not CA-ErbB2 and ROSA-flox-rtTA alone (FIG. 4E′, red). Inset in FIG. 4D′ shows co-localization. Scale bar: 20 microns. FIG. 4F. A schematic of experimental design depicts the timing of tamoxifen (amber), dox (pink), and EdU (black) injections into pups. FIGS. 4G, 4G′, 4G″, 4H, 4H′, 4H″, 4I, 4I′, and 4I″. Mice were treated as shown in (FIG. 4F). P3 cochleae were isolated and analyzed for phosphor-ERBB2 (FIGS. 4G, and 4G″, red) and SOX2 protein (FIGS. 4G′, and 4G″, cyan). All mouse genotypes harbor the ROSA-flox-rtTA modification in addition to those noted at left: CA-ErbB2 only (FIGS. 4G, 4G′, and 4G″), Sox2-CreERT/CaErbB2 (FIGS. 4H, 4H′, and 4H″), and Fgfr3-iCre/CA-ErbB2 (FIGS. 4I, 4I′ and 4I″). SOX2+ cells were reduced in number after CA-ERBB2 activation: blinded quantification found 212±62, 74±9, and 49±2 SOX2+ cells/200 microns for each genotype respectively (n=6 per genotype, ANOVA, p=0.01). Scale bar 100 microns.

FIGS. 5A, 5A′, 5A″, 5B, 5B′, 5B″, 5C, 5C′, 5C″, 5D, and 5E are a set of diagrams and photographs showing activation of CA-ErbB2 in vivo does not drive significant proliferation. FIGS. 5A, 5A′, 5A″, 5B, 5B′, 5B″, 5C, 5C′, and 5C″. Mice were treated as shown in (FIG. 4F). P3 cochleae were isolated and analyzed for SOX2 (cyan), p-ERBB2 (red) and EdU (white). All mouse genotypes harbor the ROSA-flox-rtTA modification in addition to those noted at left: CA-ErbB2 only (FIGS. 5A, 5A′, and 5A″), Sox2-CreERT/CaErbB2 (FIGS. 5B, 5B′, and 5B″), and Fgfr3-iCre/CA-ErbB2 (FIGS. 5C, 5C′, and 5C″). Scale bar: 50 microns. FIG. 5D. Fgfr3-iCre/CA-ErbB2/ROSA-flox-rtTA mice were treated as shown in (FIG. 4F), except for fixation at P14. No mice harboring Sox2-CreERT and the other transgenes survived past P6. Example confocal stack where an EdU+ cell (red fluorescence, yellow arrow) was detected in the supporting cell compartment (green) among MYO7a+ hair cells (HCs, white). Scale bar: 50 microns. FIG. 5E. Fgfr3-iCre/CA-ErbB2/ROSA-flox-rtTA mice were treated with the schedule shown in (FIG. 4F) and fixed at P8 or P14. Blinded quantification shows little difference in numbers of EdU+ cells between genotypes (n=4, 3 for each genotype, P8 and P14 respectively; ANOVA p-values not significant).

FIGS. 6A, 6B, 6C, 6D, 6E, 6F, 6G, 6H, and 6I are a set of diagrams and photographs showing activation of CA-ERBB2 in cochlear SCs at neonatal stages drives the formation of supernumerary HC-like cells in vivo. Mice were injected with tamoxifen, dox and EdU as shown previously and allowed to survive to P8 and P14, when they were analyzed for hair cell markers. Examples of supernumerary MYO7+ cells near IHCs (FIG. 6A, arrow) and OHCs (FIG. 6B) are shown in whole mount confocal stacks. Scale bar: 50 microns. Supernumerary MYO7+ cells were quantified on blinded stacks (FIG. 6C). Overall, significantly more supernumerary MYO7+ cells were observed in Fgfr3-iCre/CA-ErbB2 mice at P8 compared to Fgfr3-iCre mice alone (student's two-tailed t-test, p=0.02, n=4). Quantification of supernumerary Myo7+ cells near OHCs (top graph) and near IHCs (bottom graph) are shown. Panels (FIGS. 6D-6I) depict P14 cochleae of animals with ROSA-rtTA-GFP transgenes, and additional genotype details are indicated on the left side of the panels. HCs in control cochleae (FIGS. 6D and 6E) are revealed with MYO7 (red) and OCM (purple) immunoreactivity, near GFP+ SCs (green). Cochleae harboring activated CA-ErbB2 are also depicted (FIGS. 6F, 6G, 6H, and 6I). Supernumerary MYO7+ cells (red) co-localize with anti-OCM (purple) and anti-PVALB (FIG. 6H, cyan), indicated with yellow arrows. Both mid-base (FIGS. 6D, 6F) and apical (FIGS. 6E, 6G, 6H, and 6I) turns are shown. Supernumerary MYO7+ cells do not co-localize with SOX2 (FIG. 6I, cyan, arrow). Scale bar: 50 microns.

FIGS. 7A, 7A′, and 7B are a set of diagrams and photographs showing WS3 or WS6 treatment enhances MYO7+ cell generation in vitro. FIG. 7A. Explant cultures of the organ of Corti from postnatal mice (P1-P2) cultured for 48-72 hours in the presence of DMSO, WS3 (0.01 μM) or WS6 (0.5 μM) had extra MYO7+ cells (red) in the outer HC region, images from the apex region. SOX2, blue; Atoh1-GFP, green. (FIG. 7A′) Cross section of the organ of Corti at the yellow line in (FIG. 7A). Scale bar: 25 microns. FIG. 7B. MYO7+ cell counts in the apex, mid-apex, mid-base and base region. Significantly more MYO7+ cells were observed in the WS3 or WS6-treated cochlea than in the controls at the apex region (mean±SD per 100 μm, One-way ANOVA followed by Dunnett multiple-comparisons test, WS6 vs control p=0.0043, WS3 vs control p=0.0060, n=3-4 explants per group).

FIGS. 8A, 8A′. 8B, and 8C are a set of diagrams and photographs showing that WS3 or WS6 treatment enhanced SC proliferation in vitro. FIG. 8A. Images of the apex region of the P1-P2 organ of Corti cultured for 48-72 hours in the presence of DMSO, WS3 (0.01 μM), or WS6 (0.5 μM). MYO7a (green), SOX2 (red) and EdU (blue) are shown. IHC, inner hair cells; OHC, outer hair cells (FIG. 8A′) Cross section of organ of Corti from FIG. 8A at the yellow line. Scale bar: 25 microns. FIG. 8B. Quantification of EdU+ cells in the SC region, showing significantly more SCs in the WS3 or WS6-treated cochlea (mean±SD per 100 μm, One-way ANOVA followed by Dunnett multiple-comparisons test, WS6 vs control p=0.0053 (apex), p=0.0004 (apex-mid), p=0.0072 (mid-base), WS3 vs control p=0.0013, p=0.0002 (apex-mid), p=0.0021 (mid-base), n=3 per group). FIG. 8C. Western blot analysis of ERBB2 pathway activation was conducted using anti-p-ERBB2 antibody (Y1248) in MCF-7 cells. Cells were treated with WS3 or WS6 for 15 minutes.

FIG. 9 is a diagram showing a summary of findings from activation of ERBB family proteins in SCs. Three different methods of activation were employed: in vitro viral transduction, in vivo transgene induction, and in vitro drug manipulation. The first two methods employed cell lineage tracing (bright green) to determine the relationship between CA-ERBB2 expression and subsequent regeneration-like activities (SC proliferation and supernumerary MYO7 induction). In the third method, ERBB3 activation is presumed throughout SCs (light green). Downregulation of SOX2 protein was observed in cells neighboring transduced cells (cyan changes to grey). Proliferation was observed among SCs in both in vitro experiments, but not in vivo. MYO7 induction was observed in vivo and after drug manipulation.

FIGS. 10A, 10B, and 10C are (FIG. 10A) a diagram of the human ear indicating the flow of sound vibrations (red arrows) from the outer ear (orange) through the middle ear (pink), through the round window, and into the cochlea (blue); (FIG. 10B) a cross sectional diagram of cells of the organ of Corti, including hair cells (pink), supporting cells (brown), spiral ganglion neurons (green), stria vascularis (cyan) and lateral wall (blue). Fluid compartments are labeled to provide the orientation of the organ of Corti in comparison with (FIG. 10A); and (FIG. 10C) an electron micrograph of the surface of the organ of Corti, revealing stereocilia from outer hair cells and inner hair cells.

FIG. 11 shows the structures of WS3 and WS6.

FIG. 12 is a diagram showing noise exposure and transient CA-ERBB2 expression in supporting cells of 2.5 M old mice affect mRNA expression. Gene expression was compared by real time qPCR between no noise and noise conditions, among 2.5 M old control (Fg+/− or E+/−) and Fg+/E+ animals. Analysis focused on 2 hair cell specific transcription factor genes: Atoh1, Pou3f4; and 3 regenerative pathways: Notch, Wnt, ErbB. Gene expression was normalized to Gapdh and no noise control (Fg+/− or E+/−). Normalized gene expression from individual animals (Fold change 2ΔΔCT) was constructed into heat map.

FIGS. 13A, 13B, 13C, and 13D are a set of diagrams showing transient CA-ERBB2 expression in SCs of 2.5 M old mice alters mRNA differently under normal and noise conditions. Average gene expression (Fold change 2^{ΔΔCT}) was summarized in 4 categories: under no noise (FIG. 13A) or Noise (FIG. 13B) conditions; from 2.5 M old control (Fg+/− or E+/−) (FIG. 13C) or Fg+/E+ animals (FIG. 13D). Statistics were done by Mann-Whitney U test, *p<0.05.

FIGS. 14A, 14B, 14C, 14D, and 14E are a set of diagrams showing CA-ERBB2 expression in SCs of 1 M old mice does not affect long-term hearing. FIG. 14A. A schematic of experiments: CA-ERBB2 activation in adult mice at 1 M old and measurement of the long-term effects on hearing. DPT: Days Post Tamoxifen (Tam). FIG. 14B. Auditory brainstem response (ABR) thresholds for control mice were measured at 5 frequencies: 8, 12, 16, 24 and 32 kHz after transient CA-ERBB2. Control n=6. FIG. 14C. ABR thresholds for CA-ERBB2 transgenic mice were measured at 5 frequencies: 8, 12, 16, 24 and 32 kHz after transient CA-ERBB2. F+/E+ n=4. FIG. 14D. DPOAE thresholds for control mice were measured at 5 frequencies: 8, 12, 16, 24 and 32 kHz after transient CA-ERBB2. Control n=6. FIG. 14E. DPOAE thresholds for CA-ERBB2 transgenic mice were measured at 5 frequencies: 8, 12, 16, 24 and 32 kHz after transient CA-ERBB2. F+/E+ n=4.

FIGS. 15A, 15B, 15C, 15D, and 15E are a set of diagrams showing CA-ERBB2 expression in SCs following noise exposure may promote partial ABR recovery at 16-24 kHz in 2-3 months. FIG. 15A. A schematic of experiments: noise exposure followed by CA-ERBB2 activation in adult mice at 1 M old and measurement of the long-term effects on hearing. FIGS. 15B-15E. ABR (FIGS. 15B, 15C) and DPOAE (FIGS. 15D, 15E) thresholds for control (FIGS. 15B, 15D) and CA-ERBB2 transgenic (FIGS. 15C, 15E) mice were measured at five frequencies: 8, 12, 16, 24 and 32 kHz before and after noise (octave 8-16 kHz band, at 110 dB for 2 hours) followed by transient CA-ERBB2. Control n=10 (90DPN: n=4); F+/E+ n=6 (90DPN: n=3) Baseline: initial hearing test before start of experiments. 1, 30, 60, 90 DPN: 1, 30, 60, 90 Days Post Noise.

FIG. 16 is a set of diagrams showing an example of 24 kHz ABR waves recorded at pre-test, 1 month and 3 months after Noise exposure and CA-ERBB2 treatment. 1 F+/E+ animal and 1 control animal from the same experiment were compared. Red traces highlighted the peaks of ABR potential. ABR Threshold was decided when the peaks disappeared. All hearing tests were scored by an individual blinded to genotype and time point.

FIGS. 17A, 17B, 17C, 17D, and 17E are a set of diagrams showing averages of the ABR results from the CA-ERBB2 and control mice.

FIGS. 18A, 18B, 18C, and 18D are a set of diagrams showing hearing (threshold) recovery for control mice, ERBB mice, and CA-ERBB2 mice.

### DETAILED DESCRIPTION OF THE INVENTION

This invention is based, at least in part, on an unexpected discovery that a class of activators of the epidermal growth factor receptor (EGFR/ERBB) family of receptor tyrosine kinases signaling pathway induced expansion of various inner ear cells and their differentiation.

The loss of cochlear hair cells causes permanent hearing impairment in mammals because these crucial cells do not regenerate. In other vertebrates, hair cells differentiate from adjacent supporting cells through unknown mechanisms. Here this invention assesses the effects of activating ERBB family signaling in supporting cells. It was found that this manipulation drives generation of supernumerary MYO7+ cells in vivo, implicating it in regeneration. Surprisingly, only the neighbors of supporting cells with active signaling adopt new fates, suggesting a new model where an interplay of cell signaling involving ERBB2 regulates regeneration by neighboring stem cells. It was also shown that small molecules could mimic these effects, supporting an extension of these results to other species.

### Hearing Loss and Restoration

About one in eight adults has hearing loss, and the likelihood of hearing loss increases as one's age advances. Environmental insults that damage hearing are well known. For example, hearing loss may develop after exposure to prolonged and excessive noise or to ototoxic drugs such as aminoglycosides or platinum-containing chemotherapies. The NIDCD estimates that 26 million adult Americans have noise-induced hearing loss (NIHL, NIDCD. Quick Statistics on Hearing Loss Bethesda, Md.: National Institute of Health; 2010 [updated Jun. 16, 2010; cited 2010]), including 900,000 disabled US veterans. The Veterans' Administration spent more than $1.6 billion on annual disability payments and hearing devices in 2010. NIHL impacts speech comprehension in a noisy environment, affecting job performance and social interactions in public spaces. In spite of the financial and human costs of hearing loss from all causes, no biological treatments address its base dysfunction, namely, the damage and destruction of the sensory cells of the cochlea.

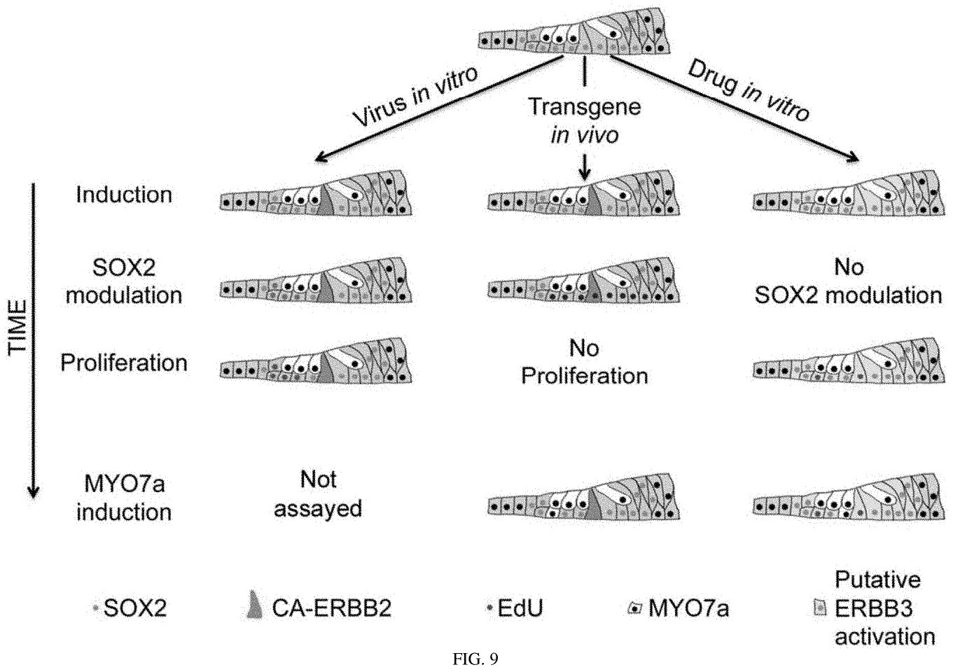

Sound enters the external auditory canal and drives vibration of the tympanic membrane (FIG. 10). This vibration is transferred to the oval window of the cochlea via three small bones, called the malleus, incus and stapes. The vibrations travel through a column of liquid called perilymph within the scala vestibuli, and will terminate at different places along the cochlea depending on their wavelength. High frequency sounds impact closer to the oval window, in the base of the cochlea, whereas low frequency sounds impact farther in, towards the cochlear apex (FIG. 10). Sensory cells line the length of the mammalian cochlea, in a region termed the organ of Corti. In the human organ of Corti, there are about 16000 sensory hair cells (3500 inner hair cells, and 12000 outer hair cells). FIG. 10B shows a schematic of a cross-section of the organ of Corti, while FIG. 10C displays an electron micrograph of its surface. Three rows of outer hair cells amplify acoustic vibrations of the tectorial membrane to promote inner hair cell activation. Inner hair cells detect these vibrations and transmit the information to spiral ganglion neurons, which signal to neurons of the cochlear nucleus in the brain. Outer hair cells, inner hair cells and spiral ganglion neurons continuously detect and transmit acoustic information throughout the life of the animal.

No regeneration has been reported for lost cells in the adult organ of Corti. Consequently, any cellular losses will persist and accumulate through the lifetime of the mammal. Noise exposure, particularly loud, low-frequency sounds, can destroy outer hair cells in the basal turn (Schuknecht H F and Gacek M R. Ann Otol Rhinol Laryngol. 1993; 102(1 Pt 2):1-16), termed noise-induced hearing loss. Such injuries are a common finding in post-mortem cochlear histology of decedents with hearing loss (Crowe S J, Guild S R, Polvogt L M. Observations on the pathology of high-tone deafness. Bulletin of the Johns Hopkins Hospital. 1934; 54(5):315. Juers A L. Clinical observations on end-organ deafness; a correlation with cochlear anatomy. Laryngoscope. 1954; 64(3):190-207. Epub 1954/03/01 and Soucek S, Michaels L, Frohlich A. Evidence for hair cell degeneration as the primary lesion in hearing loss of the elderly. J Otolaryngol. 1986; 15:175-83).

Current therapies for hearing loss rely on prosthetics, including hearing aids and cochlear implants (Groves A K. The challenge of hair cell regeneration. Experimental Biology and Medicine. 2010; 235(4):434-46). Hearing aids amplify sounds, thus counteracting the threshold shift caused by loss of outer hair cells, but still require intact inner hair cells to be innervated by auditory neurons. Cochlear implants consist of a linear array of electrodes that can directly stimulate auditory neurons. The cochlear implant is placed into patient's cochlea by surgery, and may be beneficial for profoundly deaf patients. Those methods have been shown to be successful, but there are some major limitations, such as difficulties for the patient to discriminate meaningful sounds against background noise or to hear music. Although the cochlear implant can bypass the organ of Corti machinery to artificially stimulate auditory nerves, such intervention has a chance to destroy the patient's residual hearing as well as cause auditory nerve degeneration (Brigande J V, Heller S. Quo vadis, hair cell regeneration? Nat Neurosci. 2009; 12(6):679-85).

In striking contrast to mammals, the avian auditory organ, called the basilar papilla, can regenerate lost hair cells and regain hearing function. Quiescent avian supporting cells can either directly differentiate into new hair cells, or asymmetrically divide, generating both new hair cells and supporting cells when hair cells are damaged (Corwin J T, Cotanche D A. Regeneration of sensory hair cells after acoustic trauma. Science. 1988; 240(4860):1772-4; Ryals B M, Rubel E W. Hair cell regeneration after acoustic trauma in adult Coturnix quail. Science. 1988; 240(4860):1774-6; and Stone J S, Cotanche D A. Hair cell regeneration in the avian auditory epithelium. Int J Dev Biol. 2007; 51(6-7):633-47). The regenerative capacities from avian supporting cells resemble self-renewal features of mammalian tissue-specific stem cells. The timeframe of avian regeneration is important to consider. Supporting cell proliferation and specification of new hair cells occurs within a week of deafening (Brignull H R, Raible D W, Stone J S. Feathers and fins: non-mammalian models for hair cell regeneration. Brain Res. 2009; 1277:12-23). However, the restoration of hearing thresholds can take from four to eight weeks, depending on the frequency (Ryals B M, Dent M L, Dooling R J. Return of function after hair cell regeneration. Hear Res. 2013; 297:113-20).

### ERBB Family Signaling

The ERBB receptor family is a subclass of receptor tyrosine kinases (RTKs), comprising of transmembrane glycoprotein. Ligand binding can promote dimerization among ERBB receptors, resulting in the autophosphorylation of different tyrosine residues at the intracellular domain. This family consists of four receptors: ERBB1, ERBB2, ERBB3, and ERBB4. For humans, these are commonly referred to as HER1, HER2, HER3, and HER4. ERBB1 (EGFR) and ERBB4 can homo-dimerize in a similar manner upon ligand binding. On the other hand, ERBB2 lacks the extracellular ligand-binding domain, whereas ERBB3 has an inactive tyrosine kinase domain. Hence, ERBB2 and ERBB3 cannot form homo-dimers to convey the signals. Although no known ligand binds with ERBB2, ERBB2 can hetero-dimerize with the other family members to increase their ligand binding affinity. The complexity of the signaling comes from a variety of ligands, such as Epidermal growth factor (EGF)-like ligands: neuregulin (NRG)1-4, Transforming growth factor (TGF)-α, and Heparin-binding EGF-like growth factor (HB-EGF). Different combinations of dimers can also initiate a variety of signaling cascades (Yarden Y, Sliwkowski M X. Untangling the ErbB signalling network. Nat Rev Mol Cell Biol. 2001; 2(2):127-37). Major downstream pathways from ERBB2 activation include: Mitogen-activation protein kinase (MAPK), Extracellular signal-regulated kinase (Erk) 1/2, Signal transducer and activator of transcription (STAT) and phosphatidylinositol-3-kinase (PI3K)/Protein kinase B (AKT). These pathways could promote cell proliferation and survival through mammalian target of rapamycin (mTOR) activation or p27^{Kip1} inactivation (Citri A, Skaria K B, Yarden Y. The deaf and the dumb: the biology of ErbB-2 and ErbB-3. Experimental Cell Research. 2003; 284(1):54-65).

Hair cell generation requires the basic helix-loop-helix transcription factor Atoh1 (Bermingham N A, Hassan B A, Price S D, Vollrath M A, Ben-Arie N, Eatock R A, Bellen H J, Lysakowski A, Zoghbi H Y. Math1: an essential gene for the generation of inner ear hair cells. Science. 1999; 284(5421):1837-41). Atoh1 is the earliest known hair cell lineage marker, and is active through the rest of development to ensure that hair cells properly mature (Cafaro J, Lee G S, Stone J S. Atoh1 expression defines activated progenitors and differentiating hair cells during avian hair cell regeneration. Dev Dyn. 2007; 236(1):156-70). Deletion of Atoh1 during hair cell specification results in their death (Cai T, Seymour M L, Zhang H, Pereira F A, Groves A K. Conditional Deletion of Atoh1 Reveals Distinct Critical Periods for Survival and Function of Hair Cells in the Organ of Corti. The Journal of Neuroscience. 2013; 33(24):10110-22, and Chonko K T, Jahan I, Stone J, Wright M C, Fujiyama T, Hoshino M, Fritzsch B, Maricich S M. Atoh1 directs hair cell differentiation and survival in the late embryonic mouse inner ear. Developmental Biology. 2013; 381(2):401-10).

Atoh1 expression is likely down regulated in supporting cells by from Notch lateral inhibition, which is mediated by the downstream effector Hes/Hey family of transcription factors (Hayashi T, Kokubo H, Hartman B H, Ray C A, Reh T A, Bermingham-McDonogh O. Hesr1 and Hesr2 may act as early effectors of Notch signaling in the developing cochlea. Dev Biol. 2008; 316(1):87-99). Overexpression of Atoh1 by gene transfer produces supernumerary hair cells during development (Gubbels S P, Woessner D W, Mitchell J C, Ricci A J, Brigande J V. Functional auditory hair cells produced in the mammalian cochlea by in utero gene transfer. Nature. 2008; 455(7212):537-41). Interestingly, post-mitotic supporting cells from neonatal mice still are still capable of forming hair cells following forced Atoh1 induction (Liu Z, et al., J Neurosci. 2012; 32(19):6600-10; Zheng J L, et al., Nature neuroscience. 2000; 3(6):580-6, and Kelly M C, et al., The Journal of neuroscience: the official journal of the Society for Neuroscience. 2012; 32(19):6699-710). However, this capacity declines significantly with age, and almost disappears in the mature, intact cochlea (Liu Z, et al., J Neurosci. 2012; 32(19):6600-10). Because Atoh1 plays a central role in hair cell development, many studies focus on Atoh1 overexpression to achieve hair cell regeneration in mature cochlea. Ectopic Atoh1 expression in guinea pig cochlea immediately after ototoxic injury induced immature hair cells and rescued hearing function (Izumikawa M, et al., Nat Med. 2005; 11(3):271-6). However, some following studies failed to replicate this result, possibly due to a poor correlation of the timing between the damage and Atoh1 expression (Izumikawa M, et al, Hear Res. 2008; 240(1-2):52-6 and Atkinson P J, et al, PLOS ONE. 2014; 9(7):e102077). These results indicate Atoh1 is required and sufficient to induce hair cell formation at developmental and early postnatal stage. However, overexpression of Atoh1 alone is not enough to regenerate functional hair cells in the mature cochlea.

The postnatal mammalian cochlea is mitotically quiescent. At birth (post-natal day 0 or P0), it displays a low level of regenerative capacity after hair cell ablation or toxin expression in hair cells (Cox B C, et al. Development. 2014; 141(4):816-29). Many recent studies have addressed the existence of hair cell progenitors in neonatal mouse cochlea by isolating neonatal cochlear supporting cells and culturing them in vitro (White P M, et al. Nature. 2006; 441(7096):984-7; Chai R, et al. Proc Natl Acad Sci USA. 2012; 109(21):8167-72; Shi F, et al., J Biol Chem. 2010; 285(1):392-400; and White et al. Dev Biol. 2012; 363(1):191-200). However, the capacity for endogenous regeneration declines significantly after P7. Mouse pups are born without hearing, which develops by P12; thus, studies on neonatal mouse cochlea address the capacities of the immature organ.

ERBB ligands and receptors were implicated in mouse utricular supporting cell proliferation in early experiments (Hume C R, et al., J Assoc Res Otolaryngol. 2003; 4(3):422-43 and Kuntz A L, et al., J Comp Neurol. 1998; 399(3):413-23). Unlike cochlear supporting cells, neonatal utricular supporting cells proliferate in situ in response to ERBB family ligands (Gu R, et al. Eur J Neurosci. 2007; 25(5):1363-72; Montcouquiol M, et al., J Neurosci. 2001; 21(2):570-80; and Yamashita H, et al. Proc Natl Acad Sci USA. 1995; 92(8):3152-5). Elimination of utricular hair cells can stimulate proliferation, and even regeneration, by supporting cells in vivo, although this effect is stronger in neonatal animals (Burns J C, et al., J Neurosci. 2012; 32(19):6570-7) compared to adults (Warchol M E, et al. Science. 1993; 259(5101):1619-22). ErbB ligands such as NRG-1 or TGF-β can potentiate proliferation, which is also much greater in neonatal tissue than in adults (Montcouquiol M, et al., J Neurosci. 2001; 21(2):570-80; Yamashita H, et al., Proc Natl Acad Sci USA. 1995; 92(8):3152-5; and Zheng J L, et al., J Neurocytol. 1999; 28(10-11):901-12). Proliferation only occurs in response to ligands if the utricular epithelium is removed from its mesenchymal foundation and cultured on fibronectin. NRG-1 binds to ERBB2/ERBB3 heterodimers, or to ERBB3/ERBB4 heterodimers.

In vitro, ErbB ligands promote hair cell differentiation from dissociated embryonic cochlear precursors (Doetzlhofer A, et al. Dev Biol. 2004; 272(2):432-47). Neonatal cochlear supporting cells show a latent capacity to proliferate, and are able to trans-differentiate into hair cells after purification (White P M, et al. Nature. 2006; 441(7096):984-7). Purification induces supporting cell division and down-regulation of P27^{kip1} in an age-dependent manner. Hair cells are generated from 3% of the purified supporting cell culture. 20% of newly generated hair cells incorporated Bromodeoxyuridine (BrdU), indicating mouse cochlear supporting cells can generate hair cells by both trans-differentiation and mitotic regeneration. A subset of supporting cells expressing cell surface antigen P75^{NGFR} (mainly pillar and Hensen's cells) possessed a greater potential to proliferate and generate new hair cells in vitro. A following study discovered that ERBB signaling is required in P75^{NGFR+} supporting cells for mitosis (White P M, et al. Developmental Biology. 2012; 363(1):191-200). Moreover, this requirement of ERBB signaling is conserved between bird and mammal. ERBB is necessary for cell-cycle re-entry in chicken basilar papillae to regenerate hair cells. ERBB signaling promotes the down regulation of P27^{kip1} in mouse P75^{NGFR+} supporting cells. Inhibition of ERBB signaling or of the downstream effector PI3K significantly blocks cell cycle re-entry. However, there are no reports that adding exogenous ERBB ligands affects proliferation in mouse cochlear organ cultures. Thus, from the prior literature it is unclear what role, if any, that ERBB family signaling may play in stimulating the cellular activities of cochlear regeneration.

As disclosed herein, experiments were carried out to test a candidate signaling pathway for its ability to drive the early cellular activities of cochlear regeneration: proliferation of supporting cells and their trans-differentiation into hair cells (Corwin and Cotanche 1988, Ryals and Rubel 1988, Brignull, Raible et al. 2009), with an emphasis on OHC trans-differentiation.

In these experiments multiple tools were used to drive ERBB2 signaling in mouse cochlear SCs, to test the hypothesis that ERBB2 signaling can induce either proliferation or HC differentiation in SCs. It was shown that ERBB2 signaling drives non-autonomous proliferation in neighboring SCs in vitro. The responding neighbor cells, strikingly, downregulate SOX2. It was found that new MYO7⁺ cells develop in vivo subsequent to ERBB2 activation, also in a non-cell autonomous fashion. In one example, two small molecules, WS3 and WS6, which activate ErbB signaling, were found to promote SC proliferation with increased MYO7⁺ cells in vitro. Taken together, these data are consistent with a role for ERBB receptors in a regeneration-signaling cascade, in which ERBB stimulated cells relay a short-range damage signal to endogenous cochlear stem cells to initiate a regeneration response.

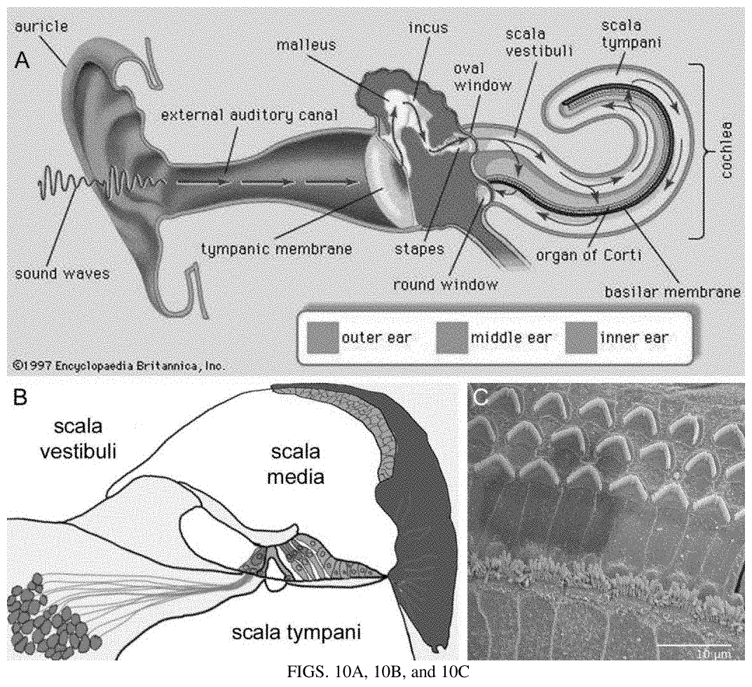

The genetic evidence described supports a role for EGF-family receptors in promoting the generation of new hair cell like cells and in mitigating hearing loss from noise in young adult mice. In some examples, two members from a diarylurea class of compounds, called WS3 and WS6, can be used in activating receptor activity and driving recovery from hearing loss (FIG. 11). These compounds have previously been shown to drive retinal pigment epithelial proliferation (Swoboda J G, et al. ACS chemical biology. 2013; 8(7):1407-11) and beta islet cell proliferation (Shen W et al., J Am Chem Soc. 2013; 135(5):1669-72) respectively. They work by diffusing into cells through the plasma membrane and inhibiting PA2G4, a negative regulator of the EGF family receptor ERBB3/HER3). PA2G4 alters ERBB3 signaling away from a mitotic signaling pathway.

### Compositions

In one aspect, the present disclosure provides a pharmaceutical composition comprising an effective amount of an inhibitor of a negative ERBB3 regulator or a pharmaceutically acceptable salt thereof. Examples of the negative ERBB3 regulator include PA2G4/EBP1, Erbb2 interacting protein (ERBIN), ERBB receptor feedback inhibitor 1 (ERRFI1) and Protein Tyrosine Phosphatase, Receptor Type K (PTPRK). Examples of PA2G4 inhibitor include WS3 (CAS #: 1421227-52-2) and WS6 (CAS #: 1421227-53-3) as well as their pharmaceutically acceptable salts. Other examples include nucleic acids, such as antisense nucleic acids and siRNAs that target PA2G4, ERBIN, ERRFI1, or PTPRK. The structures of WS3 and WS6 are described below:

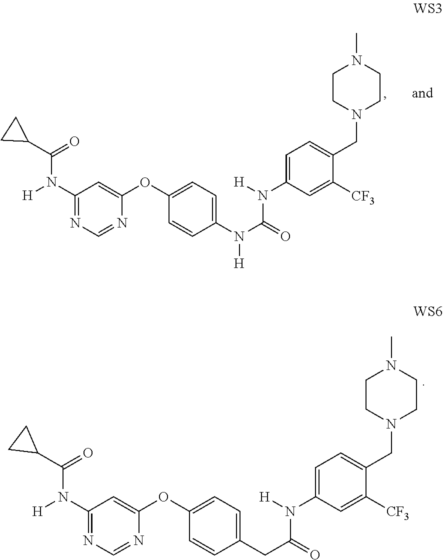 [SMILES: CN1CCN(Cc2ccc(NC(=O)Nc3ccc(Oc4cc(NC(=O)C5CC5)ncn4)cc3)cc2C(F)(F)F)CC1 ; CN1CCN(Cc2ccc(NC(=O)Cc3ccc(Oc4cc(NC(=O)C5CC5)ncn4)cc3)cc2C(F)(F)F)CC1]

In some embodiments, the composition can further comprise additional factors that can protect auditory cells before injury, preserve/promote the function of existing cells after injury, and regenerate cochlear supporting cells or hair cells after injury. Examples of these additional factors included PPAR agonists, GSK3β inhibitors, TGF-β inhibitors and differentiation inhibitors, such as, HDAC inhibitors or Notch agonists. See e.g., US20170252450, US20170071937, and US20180021320, the contents of which are incorporated by reference.

The compositions described herein can be formulated in any manner suitable for a desired delivery route, e.g., transtympanic injection, transtympanic wicks and catheters, and injectable depots. Typically, formulations include all physiologically acceptable compositions including derivatives or prodrugs, solvates, stereoisomers, racemates, or tautomers thereof with any physiologically acceptable carriers, diluents, and/or excipients.

The compositions can be used to prevent, reduce or treat the incidence and/or severity of inner ear disorders and hearing impairments involving inner ear tissue, particularly inner ear hair cells, their progenitors, and optionally, the stria vascularis, and associated auditory nerves. Of particular interest are those conditions that lead to permanent hearing loss where reduced number of hair cells may be responsible and/or decreased hair cell function. Also of interest are those arising as an unwanted side effect of ototoxic therapeutic drugs including, e.g., cisplatin and its analogs, aminoglycoside antibiotics, salicylate and its analogs, or loop diuretics. In certain embodiments, the present disclosure relates to inducing, promoting, or enhancing the growth, proliferation or regeneration of inner ear tissue, particularly inner ear supporting cells and hair cells.

The compositions are useful for the prophylaxis and/or treatment of acute and chronic ear disease and hearing loss, dizziness and balance problems especially of sudden hearing loss, acoustic trauma, hearing loss due to chronic noise exposure, presbycusis, trauma during implantation of the inner ear prosthesis (insertion trauma), dizziness due to diseases of the inner ear area, dizziness related and/or as a symptom of Meniere's disease, vertigo related and/or as a symptom of Meniere's disease, tinnitus, and hearing loss due to antibiotics and cytostatics and other drugs.

When cochlea cell populations are treated with the compositions described herein, in vivo or in vitro, the treated cells exhibit stem-like behavior in that the treated cells have the capacity to proliferate and differentiate and, more specifically, differentiate into cochlear hair cells. Alternatively, the composition induces and maintains the cells to produce daughter stem cells that can divide for many generations and maintain the ability to have a high proportion of the resulting cells differentiate into hair cells. In certain embodiments, the proliferating cells express markers which may include those disclosed in the drawings and related description below.

In some embodiments of the compositions described herein, the PA2G4 inhibitor, ERBIN inhibitor, ERRFI1 inhibitor, or PTPRK inhibitor is used at a concentration of about 1-1000 nM such as snout 5 nM to about 800 nM, about 10 nM to about 500 nM and optionally in combination with other agents.

In some embodiments, the inhibitor is an interfering nucleic acid, such as siRNA, shRNA, miRNA, antisense oligonucleotides (ASOs), and/or a nucleic acid comprising one or more modified nucleic acid residues. In some embodiments, the interfering nucleic acid is optimized (based on sequence) or chemically modified to minimize degradation prior to and/or upon delivery to the tissue of interest. Commercially available sources for these interfering nucleic acids include, but are not limited to, Thermo-Fisher Scientific/Ambion, Origene, Qiagen, Dharmacon, and Santa Cruz Biotechnology. In some embodiments, such optimizations and/or modifications may be made to assure sufficient payload of the interfering nucleic acid is delivered to the tissue of interest. Other embodiments include the use of small molecules, aptamers, or oligonucleotides designed to decrease the expression of a PA2G4, ERBIN, ERRFI1, or PTPRK gene by either binding to a gene's DNA to limit expression, e.g., antisense oligonucleotides, or impose post-transcriptional gene silencing (PTGS) through mechanisms that include, but are not limited to, binding directly to the targeted transcript or gene product or one or more other proteins in such a way that said gene's expression is reduced; or the use of other small molecule decoys that reduce the specific gene's expression.

As shown herein, the methods described herein can include reducing expression of PA2G4, ERBIN, ERRFI1, or PTPRK using inhibitory nucleic acids that target the PA2G4, ERBIN, ERRFI1, or PTPRK gene or mRNA; the sequence of the human PA2G4 cDNA is in GenBank at Acc. No. NM_006191.2 and shown below (SEQ ID NO: 1):

ATGTCGGGCGAGGACGAGCAACAGGAGCAAACTATCGCTGAGGACCTGGT
CGTGACCAAGTATAAGATGGGGGGCGACATCGCCAACAGGGTACTTCGGT
CCTTGGTGGAAGCATCTAGCTCAGGTGTGTCGGTACTGAGCCTGTGTGAG
AAAGGTGATGCCATGATTATGGAAGAAACAGGGAAAATCTTCAAGAAAGA
AAAGGAAATGAAGAAAGGTATTGCTTTTCCCACCAGCATTTCGGTAAATA
ACTGTGTATGTCACTTCTCCCCTTTGAAGAGCGACCAGGATTATATTCTC
AAGGAAGGTGACTTGGTAAAAATTGACCTTGGGGTCCATGTGGATGGCTT
CATCGCTAATGTAGCTCACACTTTTGTGGTTGATGTAGCTCAGGGGACCC
AAGTAACAGGGAGGAAAGCAGATGTTATTAAGGCAGCTCACCTTTGTGCT
GAAGCTGCCCTACGCCTGGTCAAACCTGGAAATCAGAACACACAAGTGAC
AGAAGCCTGGAACAAAGTTGCCCACTCATTTAACTGCACGCCAATAGAAG
GTATGCTGTCACACCAGTTGAAGCAGCATGTCATCGATGGAGAAAAAACC
ATTATCCAGAATCCCACAGACCAGCAGAAGAAGGACCATGAAAAAGCTGA
ATTTGAGGTACATGAAGTATATGCTGTGGATGTTCTCGTCAGCTCAGGAG
AGGGCAAGGCCAAGGATGCAGGACAGAGAACCACTATTTACAAACGAGAC
CCCTCTAAACAGTATGGACTGAAAATGAAAACTTCACGTGCCTTCTTCAG
TGAGGTGGAAAGGCGTTTTGATGCCATGCCGTTTACTTTAAGAGCATTTG
AAGATGAGAAGAAGGCTCGGATGGGTGTGGTGGAGTGCGCCAAACATGAA
CTGCTGCAACCATTTAATGTTCTCTATGAGAAGGAGGGTGAATTTGTTGC
CCAGTTTAAATTTACAGTTCTGCTCATGCCCAATGGCCCCATGCGGATAA
CCAGTGGTCCCTTCGAGCCTGACCTCTACAAGTCTGAGATGGAGGTCCAG
GATGCAGAGCTAAAGGCCCTCCTCCAGAGTTCTGCAAGTCGAAAAACCCA
GAAAAAGAAAAAAAAGAAGGCCTCCAAGACTGCAGAGAATGCCACCAGTG
GGGAAACATTAGAAGAAAATGAAGCTGGGGACTGA

The sequence of the human ERBIN mRNA/cDNA is in GenBank at Acc. No. NM_001253697.1 and shown below (SEQ ID NO: 2):

AGTTTTGTTTTTTTTTTTTTCGGCGGAGATCCTCGTTGGGGCTGGGAAACTCCTGCAAAACTCG
AGACCAGGAAGCCAGCCCGCACCCCAACCCCCACCAAAGCCACCTACTCTTCTTCTGTGGGAGG
CCAGTCCACATCCGCTCTCACCCGAGAGAGATATTCAGCTGGATCCAAAGTGACTGATGAAGGG
AAGGAAATCATGTCAAGCGAAGCCTTGAAAAAGCTGCC
CTGAGACGGTGTCCCGCCGAAAGAATGTTGGCTCAATTAAGAAACATCAGGGAGATAAATTCAA
CCCAGTGTGTCTAAAAATGACTACAAAACGAAGTTTGTTTGTGCGGTTGGTACCATGTCGCTGT
CTACGAGGGGAAGAGGAGACTGTCACTACTCTTGATTATTCTCATTGCAGCT
TAGAACAAGTTCCGAAAGAGATTTTTACTTTTGAAAAAAC CTTGGAGGAA
CTCTATTTAGATGCTAATCAGATTGAAGAGCTTCCAAAGCAACTTTTTAACTGTCAGTCTTTAC
ACAAACTGAGTTTGCCAGACAATGATTTAACAACGTTACCAGCATCCATTGCAAACCTTATTAA
TCTCAGGGAACTGGATGTCAGCAAGAATGGAATACAGGAGTTTCCAGAAAATATAAAAAATTGT
AAAGTTTTGACAATTGTGGAGGCCAGTG TAAACCCTAT
TTCCAAGCTCCCTGATGGATTTTCTCAGCTGTTAAACCTAACCCAGTTGTATCTGAAGATGCTT
TTCTTGAGTTCTTGCCAGCAAATTTTGGCAGATTAACTAAACTCCAAATATTAGAGCTTAGAGA
AAACCAGTTAAAAATGTTGCCTAAAACTATGAATAGACTGACCCAGCTGGAAAGACTGGATTTG
GGAAGTAACGAATTCACGGAAGTGCCTGAAGTACTTGAGCAACTAAGTGGATTGAAAGAGTTTT
GGATGGATGCTAATAGACTGACTTTTATTCCAGGGTTTATTGGTAGTTTGAAACAGCTCACATA
TTTGGATGTTTCTAAAAATAATATTGAAATGGTTGAAGAAGGAATTTCAACATGTGAAAACCTT
CAAGACCTCCTATTATCAAGCAATTCACTTCAGCAGCTTCCTGAGACTATTGGTTCGTTGAAGA
ATATAACAACGCTTAAAATAGATGAAAACCAGTTAATGTATCTGCCAGACTCTATAGGAGGGTT
AATATCAGTAGAAGAACTGGATTGTAGTTTCAATGAAGTTGAAGCTTTGCCTTCATCTATTGGG
CAGCTTACTAACTTAAGAACTTTTGCTGCTGATCATAATTACTTACAGCAGTTGCCCCCAGAGA
TTGGAAGCTGGAAAAATATAACTGTGCTGTTTCTCCATTCCAATAAACTTGAGACACTTCCAGA
GGAAATGGGTGATATGCAAAAATTAAAAGTCATTAATTTAAGTGATAATAGATTAAAGAATTTA
CCCTTTAGCTTTACAAAGCTACAGCAATTGACAGCTATGTGGCTCTCAGATAATCAGTCCAAAC
CCCTGATACCTCTTCAAAAAGAAACTGATTCAGAGACCCAGAAAATGGTGCTTACCAACTACAT
GTTCCCTCAACAGCCAAGGACTGAGGATGTTATGTTTATATCAGATAATGAAAGTTTTAACCCT
TCATTGTGGGAGGAACAGAGGAAACAGCGGGCTCAAGTTGCATTTGAATGTGATGAAGACAAAG
ATGAAAGGGAGGCACCTCCCAGGGAGGGAAATTTAAAAAGATATCCAACACCATACCCAGATGA
GCTTAAGAATATGGTCAAAACTGTTCAAACCATTGTACATAGATTAAAAGATGAAGAGACCAAT
GAAGACTCAGGAAGAGATTTGAAACCACATGAAGATCAACAAGATATAAATAAAGATGTGGGTG
TGAAGACCTCAGAAAGTACTACTACAGTAAAAAGCAAAGTTGATGAAAGAGAAAAATATATGAT
AGGAAACTCTGTACAGAAGATCAGTGAACCTGAAGCTGAGATTAGTCCTGGGAGTTTACCAGTG
ACTGCAAATATGAAAGCCTCTGAGAACTTGAAGCATATTGTTAACCATGATGATGTTTTTGAGG
AATCTGAAGAACTTTCTTCTGATGAAGAGATGAAAATGGCGGAGATGCGACCACCATTAATTGA
AACCTCTATTAACCAGCCAAAAGTCGTAGCACTTAGTAATAACAAAAAAGATGATACAAAGGAA
ACAGATTCTTTATCAGATGAAGTTACACACAATAGCAATCAGAATAACAGCAATTGTTCTTCTC
CATCTCGGATGTCTGATTCAGTTTCTCTTAATACTGATAGTAGTCAAGACACCTCACTCTGCTC
TCCAGTGAAACAAACTCATATTGATATTAATTCCAAAATCAGGCAAGAAGATGAAAATTTTAAC
AGCCTTTTACAAAATGGAGATATTTTAAACAGTTCAACAGAGGAAAAGTTCAAAGCTCATGATA
AAAAAGATTTTAACTTACCTGAATATGATTTGAATGTTGAAGAGCGATTAGTTCTAATTGAGAA
AAGTGTTGACTCAACAGCCACAGCTGATGACACTCACAAATTAGATCATATCAATATGAATCTT
AATAAACTTATAACTAATGATACATTTCAACCAGAGATCATGGAAAGATCAAAAACACAGGATA
TTGTGCTTGGAACAAGCTTTTTAAGCATTAATTCTAAAGAGGAAACTGAGCACTTGGAAAATGG
AAACAAGTATCCTAATTTGGAATCCGTAAATAAGGTAAATGGACATTCTGAGGAAACTTCCCAG
TCTCCTAATGGACTGAACCACATGACAGTGATTGTTCTGTTGACTTAGGTATTTCCAAAAGCAC
TGAAGATCTCTCCCCTCAGAAAAGTGGTCCAGTTGGATCTGTTGTGAAATCTCATAGCATAACT
AATATGGAGATTGGAGGGCTAAAAATCTATGATATTCTTAGTGATAATGGACCTCAGCAGCCAA
GTACAACCGTTAAAATCACATCTGCTGTTGATGGAAAAAATATAGTCAGGAGCAAGTCTGCCAC
ACTGTTGTATGATCAACCATTGCAGGTATTTACTGGTTCTTCCTCATCTTCTGATTTAATATCA
GGAACAAAGGCAATTTTCAAGTTTGATTCAAATCATAATCCCGAAGAGCCAAATATAATAAGAG
GCCCCACAAGTGGCCCACAATCTGCACCTCAAATATATGGTCCTCCACAGTATAATATCCAATA
CAGTAGCAGTGCTGCAGTCAAAGACACTTTGTGGCACTCCAAACAAAATCCCCAAATAGACCAT
GCCAGTTTTCCTCCTCAGCTCCTTCCTAGATCAGAGAGCACAGAAAATCAAAGTTATGCTAAAC
ATTCTGCCAATATGAATTTCTCTAATCATAACAATGTTCGAGCTAATACTGCATACCATTTACA
TCAGAG ACTTGGCCCA GCAAGACATG GGGAAATGTGGGCCATCTCA CCAAACGACC
GACTTATTCC TGCAGTAACT CGAAGTACAA TCCAGCGACAAAGTAGTGTG TCCTCCACAG
CCTCTGTAAA TCTTGGTGAT CCAGGCTCTA CAAGGCGGGCTCAGATTCCT GAAGGAGATT
ATTTATCATA CAGAGAGTTC CACTCAGCGG GAAGAACTCCTCCAATGATG CCAGGATCAC
AGAGACCCCT TTCTGCACGA ACATACAGCA TAGATGGTCCAAATGCATCA AGACCTCAGA
GTGCTCGACC CTCTATTAAT GAAATACCAG AGAGAACTATGTCAGTTAGT GATTTCAATT
ATTCACGGAC TAGTCCTTCA AAAAGACCAA ATGCAAGGGTTGGTTCTGAG CATTCTTTAT
TAGATCCTCC AGGAAAAAGT AAAGTTCCTC GTGACTGGAGAGAACAAGTA CTTCGACATA
TTGAAGCCAA AAAGTTAGAA AAGAAGCATC CCCAGACATCCAGTTCAGGA GATCCTTGTC
AAGATGGTAT ATTCATTTCA GGACAGCAGA ACTACTCATCAGCCACACTT AGTCACAAAG
ATGTTCCTCC AGACAGCTTG ATGAAAATGC CTTTGAGTAATGGACAGATG GGCCAGCCTC
TCAGGCCTCA GGCAAATTAT AGTCAAATAC ATCACCCCCCTCAGGCATCT GTGGCAAGGC
ATCCCTCTAG AGAACAACTA ATTGATTACT TGATGCTGAAAGTGGCCCAC CAGCCTCCAT
ATACACAGCC CCATTGTTCT CCTAGACAAG GCCATGAACTGGCAAAACAA GAGATTCGAG
TGAGGGTTGA AAAGGATCCA GAACTTGGAT TTAGCATATCAGGTGGTGTC GGGGGTAGAG
GAAACCCATT CAGACCTGAT GATGATGGTA TATTTGTAACAAGGGTACAA CCTGAAGGAC
CAGCATCAAA ATTACTGCAG CCAGGTGATA AAATTATTCAGGCTAATGGC TACAGTTTTA
TAAATATTGA ACATGGACAA GCAGTGTCCT TGCTAAAAACTTTCCAGAAT ACAGTTGAAC
TCATCATTGT ACGAGAAGTT TCCTCATAAG CACTGTGGACAAAAAAAGCG GGGAAGACAG
CAAGATTTAT TGGAAGATAC TTACAGGGGA AATTAATATTTTGACTATTT TTATATATAA
AGAAGAACTC AAAAAATTAT GTTCAAATTT GTACATTAATGAAATAATGG AACTTGTGGT
TAGAGGGAAA GAACCACTGT ACAGAATATA AAGGAGACTGTTGAATTCAT ACCATATAAA
ACTTGTTAGG TTTTTAAACA TAGCAATCAA GGCTACAAAAACAAACCTGT GTTGTTTTTG
TATAGATTGT AGGTTTATTT TTGGATTTCA TATACATGACTGAACTGTGT GCAAGGCAAT
AGTTAGCCTT GATTTTAGCC CAGAGACAGA TGGCAGAGCTATCTCTCTCA TAGCTTTTAT
GCCCTTATTT TTATTCAACT GGTATTAATG TTTTTCTCCTGAAACTACTT TTTTTGATGT
GGGCAAGAGA TTTGAAGTGT TGGCTTTTGC TATGTGCATATTGAATTGAA GAGTGAGTAG
GTGAAGGTGG TGCTGGTGGG TTCACTTTCC AAGGCCAGACTAAAACAGTT ATTTTCTATA
AAAATCTGGA AGCAAAGAAT GGGGATGGGG AGAGCTACGTGGTAGTATGT TTTTATTAGG
AGAATAATGC AATAAAATAT GTAATGTCTT TTTTATAAAGCAAAAAAGAC AATAATTGCA
TTTATGAGCT CGGCAGGATC TGTTCTTGTC ATAGCCATTGACTATACATT TGCTACTGGT
GATTCAGTTT TTAATTTTTT AGTCACAGGA AATTTTTAACTCTACTGTAG ATGCATGTCC
ATGCATTTTC TGTGTTATGG AAATCCACTG ATTTTTTTTTTTTTTTCAAA TGGTGGTACT
TGCAATCTGT TTTATAATTA GTGCTCCATT TAAATCTAATTTATAATTTT TATTTTAAGC
AGCAAATGAA ACAAAAATGG CCAGTTTTAA GATTGTGTTGCCTGTAACAC AAAATGTTAC
GAAGGTTTAG GAAAGCCTCT TTGATTTTTG TTTGGCCTTGCATTGCCTTG GTAAAGTAAA
AGGAAACAGT ACACTTGGAG CTAGGAAACC AAAGCAAGCTTTGTGAAACT GGCACAGTGA
TAGAGAATTG CTGTGGAGAG TTATAGAGCA AAGGGATGGGTCCTTGAGGC CTGCCAGTGT
GTAAAGGTGT TCAAATAAAG GGCTGTTTCT ACAGGTAACATTAAATGTGA ACTCAACACT
TCCAGAGTCT TTAAAGGGTT TCTATGTGTA TCAGTGTAATAGTGTTTTAC CACCAACTGC
CTTTCTTTGT TCCTAGTTAC TGTAACAAAT ATTTGATGATAGAGGTTTAT TAATTTTGTT
TATCCAGACC ATTAATTTTA TTTGTTTTTG TTCTATGTAATCAAATAAAA TTTGAGTAAC
ATGTAATGGT AAGGATTAAT GCATGGTTAT TTGGACCAGAAAAAAGTGCC ATAGAAGACC
AATAACTGTT TAGTTGAGGC TAGTCTGGAA CCTTTCATTAGAGCAATATT TGGTTATTGC
ACTTCATTTT TATTTACTAA GAAATGCAAT TTGGGAATTTTTAATCTGTT ATGCTTTGTT
TATCAACCTT GATTTTAATT AAGACTTTTA TAAGACTAGCTTAAAACACC AACCAACATT
ATTTTTGCAA AAGTGAGTTG GACTCACTTT CCATTCTTGCTAGTCAGAGT AAGTAGGCAG
CACTTTTAAA AATATGTGAA CTCAAATATT GCACTTCTTTCAAGATGTTA TCAATTGGTT
ATTGTACTGT ATAGTTTTAA TAATTTTGAT TGAAACCCTTTAACAACTCT TTGTAAATTT
TAACTCATTT TAGTTGATTT TCAGTACTAT TTACATAGGAATTGATTTTT ATGGATATAG
TAGAAGAAAT GTGCTGTATT TTGATAAAAT TCACTTATTGTATGTGTGTT GTAATCTAAA
AAAAAAAAGA ATGACAAACA GCTTCTTTAA GACAAGTCTCGGTGTTCCCT TTATTCTTAG
TTTGTTTTTA AATATTAATT TTGGCATTCT AAAATAGCTAACATTTCTTT TATTGATTTC
AGATTTTCAC AGGCACATTC TACTTTTAAT CAGAAATATATTTAATAAGT ATAATTGTGA
AGTTTTCAAC TACTTTACCT TGAACCACAT ATACCAATTATAATTTTGGA AAAGGAATTA
AGCCTCACGG AACAATGGAT
CTTCAGCAAACCTTAACTTCATTGTCTGCACATTACATTGAAGTATTATA
AATGCAACAGATGTTATATGCACTGGCATTTTATCCTACTCTAGTTAGTTAAAATTTTATAGTA
TTCTTGCAACACATAAAGTTGCGTAAGAAACTTTACCAAGAGGAGTATTATAGCCAAGTTTTCT
TTGAAAGTATTGGAAAACTAAAATTAAATGACAAGGACTTTGAATTAGAATTTTGCTGTAATAA
AGTTTCAAAATTTGAATAAAATAATTAAATTTTTTGAG GA

The sequence of the human ERRFI1 mRNA/cDNA is in GenBank at Ace. No. JQ867454.1 and shown below (SEQ ID NO: 3):

CTACCTCCCA GGGAATGAAA GCTACTGGTT GATTTTAAAG TGCCTGGGCC
TCACAGGTTTGGAGATGTCC CAGAATAAGG CACAATGTCA ATAGCAGGAG TTGCTGCTCA
GGAGATCAGAGTCCCATTAA AAACTGGATT TCTACATAAT GGCCGAGCCA TGGGGAATAT
GAGGAAGACCTACTGGAGCA GTCGCAGTGA GTTTAAAAAG TAAGTAGAGG ATGTAATGCT
GCTGTAATCTGGATAAATAT GTGACACTAA AATGGGAGAG GCTGTGATTG CTCTTCGCTT
ATGACCAAAGTAGCTTCCTC TCCTTTCAGC AACTTTTTAA ATATTGACCC GATAACCATG
GCCTACAGTCTGAACTCTTC TGCTCAGGAG CGCCTAATAC CACTTGGTAT GTATTCTGAA
AATCTGATCACAGTAAGCAT TTGAGAAGAA CAGTCTGGAT TCGGGTTAGC TTGTCCTCCA
GCATTATTTTTTAAATGAGG AAACCTGAAC TATTTCCAAC AACAGCCTGA CCCCTAGTGG
CAACAGATTCAGAAGATAAC TGTGTTTTTC TCAAGCTATT GTACTCGACT GCCTTCATTC
TGAGTCACTGATTGCTAAGT AGGACTGTTC ATGGACGTGG GATCTTCTAA AATCAAGAAT
TAGTTCTCATTCCAGCTCTG ATGCATACTT TACTTCATGA AACCTTAGGC GAGATTTCCC
ACCTTTCTTACTAGTATCGA ATGCATGTTT GACAGTAATA GATGAAAATA GTATAAATGT
TCCTCAAAACTTAAAAAATA GTATTTTTAA TGTGAATATT CTGTTCCTTG GATCTTTGTC
AAGAGCTGTGTGTGAACTGA ACACATTGCA GGCAAGTCCA TTCACTCACA ATATTATGAT
GGGCCAGCAATAAGGACTTT GTCTTATCTC ATTGGTACCC TACGTGCCTA GTATGGTCGC
ATGTCTTAAATGGCAAGGCT GGTACAGTAT GGTATTCATG TAAATTATAT GCTATTCATC
TTCCGCGAATTTTACACACG TCACAAAACT TGCCTGTGAT GTGTGGGTGT GCGCTGTGCA
CATGTCCAAGGGAGATAGAG GAGATAGTTT GTTCTTTGAA CCACACCATG TGCGTTAAGA
ATCTTCTGCTCTCTAATTAC ACCTGTGGTG GTTGCATGGG TGTTCTCGGG GTGACAGCAG
TCAAGTGTTTCACTCAGGAA GAAAGCTGTG GAAGCATAGG TAGCTGGGGT GCTCTCTCCC
TCACACAGGTGGAGAGAGGA TTGTTGATCT TTTATTAATA TCTCTCGTTC ATTCCAGGGC
ATGCTTCCAAATCTGCTCCG ATGAATGGCC ACTGCTTTGC AGAAAATGGT CCATCTCAAA
AGTCCAGCTTGCCCCCTCTT CTTATTCCCC CAAGTGAAAA CTTGGGACCA CATGAAGAGG
ATCAAGTTGTATGTGGTTTT AAGAAACTCA CAGTGAATGG GGTTTGTGCT TCCACCCCTC
CACTGACACCCATAAAAAAC TCCCCTTCCC TTTTCCCCTG TGCCCCTCTT TGTGAACGGG
GTTCTAGGCCTCTTCCACCG TTGCCAATCT CTGAAGCCCT CTCTCTGGAT GACACAGACT
GTGAGGTGGAATTCCTAACT AGCTCAGATA CAGACTTCCT TTTAGAAGAC TCTACACTTT
CTGATTTCAAATATGATGTT CCTGGCAGGC GAAGCTTCCG TGGGTGTGGA CAAATCAACT
ATGCATATTTTGATACCCCA GCTGTTTCTG CAGCAGATCT CAGCTATGTG TCTGACCAAA
ATGGAGGTGTCCCAGATCCA AATCCTCCTC CACCTCAGAC CCACCGAAGA TTAAGAAGGT
CTCATTCGGGACCAGCTGGC TCCTTTAACA AGCCAGCCAT AAGGATATCC AACTGTTGTA
TACACAGAGCTTCTCCTAAC TCCGATGAAG ACAAACCTGA GGTTCCCCCC AGAGTTCCCA
TACCTCCTAGACCAGTAAAG CCAGATTATA GAAGATGGTC AGCAGAAGTT ACTTCGAGCA
CCTATAGTGATGAAGACAGG CCTCCCAAAG TACCGCCAAG AGAACCTTTG TCACCGAGTA
ACTCGCGCACACCGAGTCCC AAAAGCCTTC CGTCTTACCT CAATGGGGTC ATGCCCCCGA
CACAGAGCTTTGCCCCTGAT CCCAAGTATG TCAGCAGCAA AGCACTGCAA AGACAGAACA
GCGAAGGATCTGCCAGTAAG GTTCCTTGCA TTCTGCCCAT TATTGAAAAT GGGAAGAAGG
TTAGTTCAACACATTATTAC CTACTACCTG AACGACCACC ATACCTGGAC AAATATGAAA
AATTTTTTAGGGAAGCAGAA GAAACAAATG GAGGCGCCCA AATCCAGCCA TTACCTGCTG
ACTGCGGTATATCTTCAGCC ACAGAAAAGC CAGACTCAAA AACAAAAATG GATCTGGGTG
GCCACGTGAAGCGTAAACAT TTATCCTATG TGGTTTCTCC TTAGACCTTG GGGTCATGGT
TCAGCAGAGGTTACATAGGA GCAAATGGTT CTCAATTTTC CAGTTTGATT GAAGTGCAGA
GAAAAATCCCTTA

The sequence of the human PTPRK mRNA/cDNA is in GenBank at Ace. No. BC144512.1 and shown below (SEQ ID NO: 4):

GGCTGTCCTC TCACCGTCCT CACCCCGCGA GGCCCGGCCC GCTCCTCCGT
CGTGGATTTCGCGGCGATCC CCCCGGCAGC TCTTTGCAAA GCTGCTTGAA ACTTCTCCCA
AACTCGGCATGGATACGACT GCGGCGGCGG CGCTGCCTGC TTTTGTGGCG CTCTTGCTCC
TCTCTCCTTGGCCTCTCCTG GGATCGGCCC AAGGCCAGTT CTCCGCAGGT GGCTGTACTT
TTGATGATGGTCCAGGGGCC TGTGATTACC ACCAGGATCT GTATGATGAC TTTGAATGGG
TGCATGTTAGTGCTCAAGAG CCTCATTATC TACCACCCGA GATGCCCCAA GGTTCCTATA
TGATAGTGGACTCTTCAGAT CACGACCCTG GAGAAAAAGC CAGACTTCAG CTGCCTACAA
TGAAGGAGAACGACACTCAC TGCATTGATT TCAGTTACCT ATTATATAGC CAGAAAGGAC
TGAATCCTGGCACTTTGAAC ATATTAGTTA GGGTGAATAA AGGACCTCTT GCCAATCCAA
TTTGGAATGTGACTGGATTC ACGGGTAGAG ATTGGCTTCG GGCTGAGCTA GCAGTGAGCA
CCTTTTGGCCCAATGAATAT CAGGTAATAT TTGAAGCTGA AGTCTCAGGA GGGAGAAGTG
GTTATATTGCCATTGATGAC ATCCAAGTAC TGAGTTATCC TTGTGATAAA TCTCCTCATT
TCCTCCGTCTAGGGGATGTA GAGGTGAATG CAGGGCAAAA CGCTACATTT CAGTGCATTG
CCACAGGGAGAGATGCTGTG CATAACAAGT TATGGCTCCA GAGACGAAAT GGAGAAGATA
TACCAGTAGCCCAGACTAAG AACATCAATC ATAGAAGGTT TGCCGCTTCC TTCAGATTGC
AAGAAGTGACAAAAACTGAC CAGGATTTGT ATCGCTGTGT AACTCAGTCA GAACGAGGTT
CCGGTGTGTCCAATTTTGCT CAACTTATTG TGAGAGAACC GCCAAGACCC ATTGCTCCTC
CTCAGCTTCTTGGTGTTGGG CCTACATATT TGCTGATCCA ACTAAATGCC AACTCGATCA
TTGGCGATGGTCCTATCATC CTGAAAGAAG TAGAGTACCG AATGACATCA GGATCCTGGA
CAGAAACCCATGCAGTCAAT GCTCCAACTT ACAAATTATG GCATTTAGAT CCAGATACCG
AATATGAGATCCGAGTTCTA CTTACAAGAC CTGGTGAAGG TGGAACGGGG CTCCCAGGAC
CTCCACTAATCACCAGAACA AAATGTGCAG AACCTATGAG AACCCCAAAG ACATTAAAGA
TTGCTGAAATACAGGCAAGA CGGATTGCTG TGGACTGGGA ATCCTTGGGT TACAACATTA
CGCGTTGCCACACTTTTAAT GTCACTATCT GCTACCATTA CTTCCGTGGT CACAACGAGA
GCAAGGCAGACTGTTTGGAC ATGGACCCCA AAGCCCCTCA GCATGTTGTG AACCATCTGC
CACCTTATACAAATGTCAGC CTCAAGATGA TCCTAACCAA TCCAGAGGGA AGGAAGGAGA
GTGAAGAGACAATTATTCAA ACTGATGAAG ATGTGCCTGG TCCCGTACCA GTAAAATCTC
TTCAAGGAACATCCTTTGAA AATAAGATCT TCTTGAACTG GAAAGAACCT TTGGATCCAA
ATGGAATCATCACTCAATAT GAGATCAGCT ATAGCAGTAT AAGATCATTT GATCCTGCAG
TTCCAGTGGCTGGACCTCCC CAGACTGTAT CAAATTTATG GAACAGTACA CACCATGTCT
TTATGCATCTCCACCCTGGA ACCACGTACC AGTTTTTCAT AAGAGCCAGC ACGGTCAAAG
GCTTTGGTCCAGCCACAGCC ATCAATGTCA CCACCAATAT CTCAGCTCCA ACTTTACCTG
ACTATGAAGGAGTTGATGCC TCTCTCAATG AAACTGCCAC CACAATAACT GTATTGTTGA
GACCAGCACAAGCCAAAGGT GCTCCTATCA GTGCTTATCA GATTGTTGTG GAAGAACTGC
ACCCACACCGAACCAAGAGA GAAGCCGGAG CCATGGAATG CTACCAGGTT CCTGTCACAT
ACCAAAATGCCATGAGTGGG GGTGCACCGT ATTACTTTGC TGCAGAACTA CCCCCGGGAA
ACCTACCTGAGCCTGCCCCG TTCACTGTGG GTGACAATCG GACCTACCAA GGCTTTTGGA
ACCCTCCTTTGGCTCCGCGC AAAGGATACA ACATCTATTT CCAGGCGATG AGCAGTGTGG
AGAAGGAAACTAAAACCCAG TGCGTACGCA TTGCTACAAA AGCAGCAGCA ACAGAAGAAC
CAGAAGTGATCCCAGATCCC GCCAAGCAGA CAGACAGAGT GGTGAAAATA GCAGGAATTA
GTGCTGGAATTTTGGTGTTC ATCCTCCTTC TCCTAGTTGT CATATTAATT GTAAAAAAGA
GCAAACTTGCTAAAAAACGC AAAGATGCCA TGGGGAATAC CCGGCAGGAG ATGACTCACA
TGGTGAATGCAATGGATCGA AGTTATGCTG ATCAGAGCAC TCTGCATGCA GAAGATCCTC
TTTCCATCACCTTCATGGAC CAACATAACT TTAGTCCAAG ATATGAGAAC CACAGTGCTA
CAGCAGAGTCCAGTCGCCTT CTAGACGTAC CTCGCTACCT CTGTGAGGGG ACGGAATCCC
CTTACCAGACAGGACAGCTG CATCCAGCCA TCAGGGTAGC TGATTTACTG CAGCACATTA
ATCTCATGAAGACATCAGAC AGCTATGGGT TCAAAGAGGA ATATGAGAGC TTTTTTGAAG
GACAGTCAGCATCTTGGGAT GTAGCTAAAA AAGATCAAAA TAGAGCAAAA AACCGATATG
GAAACATTATAGCATATGAT CACTCCAGAG TGATTTTGCA ACCCGTAGAG GATGATCCTT
CCTCAGATTATATTAATGCC AACTATATTG ATATTTGGCT GTACAGGGAT GGCTACCAGA
GACCAAGTCATTACATTGCA ACCCAAGGTC CCGTTCATGA AACAGTGTAT GATTTCTGGA
GGATGATTTGGCAAGAACAA TCTGCTTGCA TTGTGATGGT TACAAATTTA GTTGAGGTTG
GCCGGGTTAAATGCTATAAA TATTGGCCTG ATGATACTGA AGTTTATGGT GACTTCAAAG
TAACGTGTGTAGAAATGGAA CCACTTGCTG AATATGTAGT TAGGACATTC ACCCTGGAAA
GGAGGGGGTACAATGAAATC CGTGAAGTTA AACAGTTCCA TTTCACGGGC TGGCCTGACC
ATGGAGTGCCCTACCATGCT ACAGGGCTGC TTTCCTTTAT CCGGCGAGTC AAGTTATCAA
ACCCTCCCAGTGCTGGCCCC ATCGTTGTAC ATTGCAGTGC TGGTGCTGGA CGAACTGGCT
GCTACATTGTGATTGACATC ATGCTAGACA TGGCTGAAAG AGAGGGTGTT GTTGATATTT
ACAATTGTGTCAAAGCCTTA AGATCTCGGC GTATTAATAT GGTCCAGACA GAGGAACAGT
ACATTTTTATTCATGATGCC ATTTTAGAAG CCTGCTTATG TGGAGAAACT GCCATACCTG
TCTGTGAATTTAAAGCTGCA TATTTTGATA TGATTAGAAT AGACTCCCAG ACTAACTCTT
CACATCTCAAGGATGAATTT CAGACTCTGA ATTCAGTCAC CCCTCGACTA CAAGCTGAAG
ACTGCAGTATAGCGTGCCTG CCAAGGAACC ATGACAAGAA CCGTTTCATG GACATGCTGC
CACCTGACAGATGTCTGCCT TTTTTAATTA CAATTGATGG GGAGAGCAGT AACTACATCA
ATGCTGCTCTTATGGACAGC TACAGGCAAC CAGCTGCTTT CATCGTCACA CAATACCCTC
TGCCAAACACTGTAAAAGAC TTCTGGAGAT TAGTGTATGA TTATGGCTGT ACCTCCATTG
TGATGTTAAACGAAGTCGAC TTGTCCCAGG GCTGCCCTCA GTACTGGCCA GAGGAAGGGA
TGCTACGATATGGCCCCATC CAAGTGGAAT GTATGTCTTG TTCAATGGAC TGTGATGTGA
TCAACCGGATTTTTAGGATA TGCAATCTAA CAAGACCACA GGAAGGTTAT CTGATGGTGC
AACAGTTTCAGTACCTAGGA TGGGCTTCTC ATCGAGAAGT GCCTGGATCC AAAAGGTCAT
TCTTGAAACTGATACTTCAG GTGGAAAAGT GGCAGGAGGA ATGCGAGGAA GGGGAAGGCC
GGACGATTATCCACTGCCTA AATGGTGGCG GGCGAAGTGG CATGTTCTGT GCTATAGGCA
TCGTTGTTGAAATGGTGAAA CGGCAAAATG TTGTCGATGT TTTCCATGCA GTAAAGACAC
TGAGGAACAGCAAGCCAAAC ATGGTGGAAG CCCCGGAGCA ATACCGTTTC TGCTATGATG
TAGCTTTGGAGTACCTGGAA TCATCTTAGT TGGGTGAGAC TCTTTAAAGT GCATCCATGA
AGAAACCTGTCCATCTATTG AGCCAGCAGC TGTTGTACCT GTTACACTTG TGCAGAAAGA
TTTTAATGTGGGGGGTGGGA GACTTTTACA TTTGAGAGGT AAAAGTATTT TTTTTATGAA
GTTGTGTATCTTAATAAAAA GGACTGAATT AGTTTTTATT ACTATATTAA AGCATCAACA
TTTCATGCCACATAAATTAT ATTTAATAAG AACCAGATTG AAATGAGAAC GTATTGGTGT
TTGTACAGTGAACATGCCAC CTTTTTTCTC ATGGTTTCAG TAGAGCAGCT ACCACATGTT
GCATGAGTTCATACTTTCTA CGTGGCATTT TTCTCCCTTT CTAAAATGAA AGCTGATGAA
TCTTAAAAGGAAGAAGAAAA GAAAAGCTGT GCAAATTCAT AGTAAAGTTC GTTTTTTATA
TGTTTCCAGTGTAGCAGATC TCTATATAAA TATATAAATA TATATAACTG GCTTATTTTC
TTTTAATGTGCAATGATGGC TGGATCATTT AAAGTTCTTT TTAGAAAATA ACATAAGCCA
AAGACTCAAGTGTAAATATG TCTATATGGA GAAAGCACAT TATATTTATT GGTTACTTAC
ATTCCTTTTTTGATGGCTAA AATACTACCA CCACACAATC ATCTTTTTTT TCCTGAAGAA
AGCTTTTTCTTTAGCTAAAA TCAATTGTAA ACGATTTTTG TAGATTATTT TTTGTATGTT
TTAGTGTAAGTAGAAGATAA ACTTTTTATT CATAAACCAG GAAGCAATGT TCTTTATAGT
GATTCTCTTGTGTACATGCT TGTGAATTAA ATTTGTGTAA AATCCCTTGG CAATTGGGTC
TTTTAATATAGGACCAAATT AAAACATTTT GCTGAATATG TATAGTTTTT CACAATTTCA
TTAGGTAAATAATGGTTTGG TGATCATACA TGAGAAATGT ACACATTAAA AGGCCTTGCT
GACAACTTGCACAATGTTGA ACATAGCCTT TAAGCATCAT TTAAATTTTA AAGGAATGGA
GTTTTTCAGCCTGTGGCCCA GCACTGGTCA AGAAAACAAG ATGGCAACAT ATATGCTTTC
AGGGTCAAATTTGAGCAAAC TGTAAACTGT CAGGGTGATA AAATGTTTCT CTTGATGTTT
ACATGCACAAGCTTTGCGTT CTGACTATAA AAAGTGTGAA CAAATCAATG CCAGATTCCT
GTTTTGCGCATTGTCATGG

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target PA2G4 nucleic acid and modulate its function. In some embodiments, the inhibitory nucleic acids include antisense oligonucleotides, e.g., antisense RNA, antisense DNA, chimeric antisense oligonucleotides, or antisense oligonucleotides comprising modified linkages or nucleotide; interfering RNA (RNAi), e.g., small interfering RNA (siRNA), or a short hairpin RNA (shRNA); or combinations thereof. The inhibitory nucleic acids can be modified, e.g., to include a modified nucleotide (e.g., locked nucleic acid) or backbone (e.g., backbones that do not include a phosphorus atom therein), or can by mixmers or gapmers; see, e.g., WO2013/006619, which is incorporated herein by reference for its teachings related to modifications of oligonucleotides. Suitable siRNAs directed against PA2G4 can be obtained commercially from vendors such as Origene and Santa Cruz Biotechnology, Inc.

The pharmaceutical compositions described herein can be adapted to administer the drug locally to the round or oval membrane. To that end, the pharmaceutical compositions may also contain a membrane penetration enhancer, which supports the passage of the active ingredient through the round or oval membrane. For example, liquid, gel or foam compositions may be used. Although it is also possible to apply the active ingredient orally or to employ a combination of delivery approaches, the active ingredient (e.g., WS3 and/or WS6) is preferably administered into the organ of Corti to limit the scope of affected tissues.

### Administration

The above-described the inhibitor or composition can be administered by any suitable means known in the art, including intratympanic administration and intracochlear administration using microneedle/syringe, nanoparticles, cell-penetrating peptides, magnetic force, gel, ear cube, viral vectors, and apical injections. See e.g., Hao et al. Eur J Pharm Sci. 2018 May 21. pii: S0928-0987(18)30239-2.

In one embodiment, intratympanic or intracochlear delivery of drugs can be used in a sustained manner using microcatheters and microwicks. Alternatively, the drugs can be applied as single or as repeated injections (e.g., 1-8 injections over periods of up to 1-2 weeks). Intratympanically applied drugs are thought to enter the fluids of the inner ear primarily by crossing the round or oval (RW) membrane. The volume of inner ear fluids is very small, on the order of 10 μl. The inventors have observed effects from WS3 application in culture at, e.g., 10 nM, and for WS6 at, e.g., 500 nM. The molecular weight of WS3 is 280 g/mole, and WS6 is 569 g/mole. Because these compounds would be locally applied, the dosages can be very small, such as 0.01-1 ng/injection for WS3, and 0.3-30 ng/injection for WS6.

Calculations show that a major factor controlling both the amount of drug entering the ear and the distribution of drug along the length of the ear is the duration the drug remains in the middle ear space. Single, “one-shot” applications or applications of aqueous solutions for few hours' duration result in steep drug gradients for the applied substance along the length of the cochlea and rapidly declining concentration in the basal turn of the cochlea as the drug subsequently becomes distributed throughout the ear.

In a preferred embodiment, the drug (e.g., WS3 and/or WS6) is be injected into the organ of Corti to limit the scope of affected tissues. The drug may be injected into either the scala tympani or the scala media (see FIG. 10). The former may be accessed through the round window, whereas the latter requires surgical techniques including cochleostomy or canalostomy. In that case, one can administer the drug in a sponge, gel, biopolymer, tubing, or pump to the round window, enabling the compounds to diffuse through it and enter the organ of Corti.

Exemplary injection approaches include by osmotic pump, or, by combination with implanted biomaterial, by injection or infusion. Biomaterials that can aid in controlling release kinetics and distribution of drug include hydrogel materials, degradable materials. One class of materials that is used includes in situ gelling materials. All potential materials and methodologies mentioned in these references are included herein by reference (Almeida H, Amaral M H, Lobao P, Lobo J M. In situ gelling systems: a strategy to improve the bioavailability of ophthalmic pharmaceutical compositions. Drug Discovery Today 2014; 19:400-12; Wise A K, Gillespie L N. Drug delivery to the inner ear. Journal of Neural Engineering 2012; 9:065002; Surovtseva E V, Johnston A H, Zhang W, et al. Prestin binding peptides as ligands for targeted polymersome mediated drug delivery to outer hair cells in the inner ear. International Journal of Pharmaceutics 2012; 424:121-7; Roy S, Glueckert R, Johnston A H, et al. Strategies for drug delivery to the human inner ear by multifunctional nanoparticles. Nanomedicine 2012; 7:55-63; Rivera T, Sanz L, Camarero G, Varela-Nieto I. Drug delivery to the inner ear: strategies and their therapeutic implications for sensorineural hearing loss. Current Drug Delivery 2012; 9:231-42; Pararas E E, Borkholder D A, Borenstein J T. Microsystems technologies for drug delivery to the inner ear. Advanced drug delivery reviews 2012; 64:1650-60; Li M L, Lee L C, Cheng Y R, et al. A novel aerosol-mediated drug delivery system for inner ear therapy: intratympanic aerosol methylprednisolone can attenuate acoustic trauma. IEEE Transactions on Biomedical Engineering 2013; 60:2450-60; Lajud S A, Han Z, Chi F L, et al. A regulated delivery system for inner ear drug application. Journal of controlled release: official journal of the Controlled Release Society 2013; 166:268-76; Kim D K, Park S N, Park K H, et al. Development of a drug delivery system for the inner ear using poly(amino acid)-based nanoparticles. Drug delivery 2014; Kanzaki S, Fujioka M, Yasuda A, et al., PloS ONE 2012; 7:e48480; Engleder E, Honeder C, Klobasa J, Wirth M, Arnoldner C, Gabor F. Preclinical evaluation of thermoreversible triamcinolone acetonide hydrogels for drug delivery to the inner ear. International Journal of Pharmaceutics 2014; 471:297-302; Bohl A, Rohm H W, Ceschi P, et al. Development of a specially tailored local drug delivery system for the prevention of fibrosis after insertion of cochlear implants into the inner ear. Journal of Materials Science: Materials in Medicine 2012; 23:2151-62; Hoskison E, Daniel M, Al-Zahid S, Shakesheff K M, Bayston R, Birchall J P. Drug delivery to the ear. Therapeutic Delivery 2013; 4:115-24; Staecker H, Rodgers B., Expert Opin Drug Deliv 2013; 10:639-50; Pritz C O, Dudas J, Rask-Andersen H, Schrott-Fischer A, Glueckert R. Nanomedicine strategies for drug delivery to the ear. Nanomedicine 2013; 8:1155-72), which are included herein by reference in their entirety. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high speed jet.

When used for human and veterinary treatment, the amount of a particular agent that is administered may be dependent on a variety of factors, Examples of these factors include the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

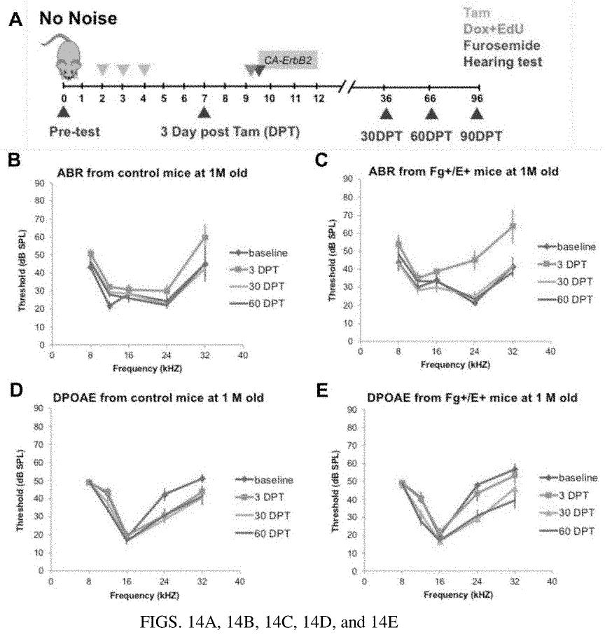

The inventors show here that activating CA-ERBB2 in supporting cells does not have long-lasting effects on hearing in the absence of noise (FIG. 14). However, peripheral glial cells respond to NRG1 by re-entering the cell cycle in an ERBB3 dependent process. Unregulated glial cell proliferation can generate a schwannoma, or glial tumor. Eighth nerve schwannomas are typically treated by removing the inner ear, which would be a poor outcome for a treatment for hearing loss.

The agents described herein may be administered in a therapeutically effective amount to a subject in need of treatment. Administration of compositions described herein can be via any of suitable route of administration, particularly by intratympanically. Other routes may include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical by ear instillation for absorption through the skin of the ear canal and membranes of the eardrum. Such administration may be as a single or multiple oral doses, defined number of eardrops, or a bolus injection, multiple injections, or as a short- or long-duration infusion. Implantable devices (e.g., implantable infusion pumps) may also be employed for the periodic parenteral delivery over time of equivalent or varying dosages of the particular composition. For such parenteral administration, the compositions are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives. The preparation of suitable and sterile parenteral compositions is described in detail in the section entitled “Compositions” above.

Compositions described herein can be administered by a number of methods sufficient to deliver the composition to the inner ear. Delivering a composition to the inner ear includes administering the composition to the middle ear, such that the composition may diffuse across the round or oval to the inner ear and administering a composition to the inner ear by direct injection through the round or oval membrane. Such methods include, but are not limited to auricular administration, by transtympanic wicks or catheters, or parenteral administration, for example, by intraauricular, transtympanic, or intracochlear injection. In particular embodiments, the compositions and compositions of the disclosure are locally administered, not administered systemically.

In one embodiment, a syringe and needle apparatus can be used to administer compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round or oval or immediately adjacent to the round or oval. Exemplary devices used for auricular administration include, but are not limited to, transtympanic wicks, transtympanic catheters, round or oval microcatheters (small catheters that deliver medicine to the round or oval), and SILVERSTEIN MICROWICKS (small tube with a “wick” through the tube to the round or oval, allowing regulation by subject or medical professional).

In another embodiment, a syringe and needle apparatus can be used to administer compositions to a subject using transtympanic injection, injection behind the tympanic membrane into the middle and/or inner ear. The composition may be administered directly onto the round or oval membrane via transtympanic injection or may be administered directly to the cochlea via intracochlear injection or directly to the vestibular organs via intravestibular injection.

In some embodiments, the delivery device can be an apparatus designed for administration of compositions to the middle and/or inner ear. Examples include those marketed by GYRUS Medical Gmbh, which have micro-otoscopes for visualization of and drug delivery to the round or oval niche, and devices to deliver fluids to inner ear structures described in in U.S. Pat. Nos. 6,045,528, 5,421,818, 5,474,529, 5,476,446, and US 2007/0167918, each of which is incorporated by reference herein for such disclosure.

In some embodiments, a composition disclosed herein is administered to a subject in need thereof once. In some embodiments, a composition disclosed herein is administered to a subject in need thereof more than once. In some embodiments, a first administration of a composition disclosed herein is followed by a second, third, fourth, or fifth administration of a composition disclosed herein.

The frequency or number of times a composition is administered to a subject in need thereof depends on the discretion of a medical professional, the disorder, the severity of the disorder, and the subject's response to the composition. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; alternatively, the dose of drug being administered may be temporarily reduced or temporarily suspended for a certain length of time. Once the subject's hearing and/or balance has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

### Kits/Articles of Manufacture

The disclosure also provides kits for preventing or treating hearing loss and/or preventing or inhibiting hair cell degeneration or hair cell death in a subject, preferably in human. Such kits generally will comprise one or more EBP inhibitors or the pharmaceutical composition disclosed herein, and instructions for using the kit. The disclosure also contemplates the use of one or more EBP inhibitors or the pharmaceutical composition disclosed herein, in the manufacture of medicaments for treating, abating, reducing, or ameliorating the symptoms of a disease, dysfunction, or disorder in a mammal, such as a human that has, is suspected of having, or at risk for developing hearing loss, hair cell degeneration or hair cell death.

In some embodiments, the kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein generally will comprise one or more EBP inhibitors or the pharmaceutical composition disclosed herein and packaging materials. Examples of pharmaceutical packaging materials include, but are not limited to, blister packs, bottles, tubes, inhalers, pumps, bags, vials, containers, syringes, bottles, and any packaging material suitable for a selected composition and intended mode of administration and treatment.

### Definition

In this application, the use of “or” means “and/or” unless stated otherwise. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers to introducing a substance into a subject. In some embodiments, administration is auricular, intraauricular, intracochlear, intravestibular, or transtympanically, e.g., by injection. In some embodiments, administration is directly to the inner ear, e.g., injection through the round or oval, otic capsule, or vestibular canals. In some embodiments, administration is directly into the inner ear via a cochlear implant delivery system. In some embodiments, the substance is injected transtympanically to the middle ear. In certain embodiments “causing to be administered” refers to administration of a second component after a first component has already been administered (e.g., at a different time and/or by a different actor).

“Auricular administration” refers to a method of using a catheter or wick device to administer a composition across the tympanic membrane to the inner ear of the subject. To facilitate insertion of the wick or catheter, the tympanic membrane may be pierced using a suitably sized syringe or pipette. The devices could also be inserted using any other methods known to those of skill in the art, e.g., surgical implantation of the device. In particular embodiments, the wick or catheter device may be a stand-alone device, meaning that it is inserted into the ear of the subject and then the composition is controllably released to the inner ear. In other particular embodiments, the wick or catheter device may be attached or coupled to a pump or other device that allows for the administration of additional compositions. The pump may be automatically programmed to deliver dosage units or may be controlled by the subject or medical professional.

“Anti-sense” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

An “inhibitor” refers to an agent that causes a decrease in the expression or activity of a target gene or protein, respectively. An “antagonist” can be an inhibitor, but is more specifically an agent that binds to a receptor, and which in turn decreases or eliminates binding by other molecules.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

“Population” of cells refers to any number of cells greater than 1, but is at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), microRNA (mRNA) and/or nucleic acids (such as siRNA, shRNA, or miRNA) comprising one or more modified nucleic acid residue—e.g. peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

“Subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like. “Mammal” refers to any mammal including but not limited to human, mouse, rat, sheep, monkey, goat, rabbit, hamster, horse, cow or pig.

“Supporting Cell” as used herein in connection with a cochlear epithelium comprises epithelial cells within the organ of Corti that are not hair cells. This includes inner pillar cells, outer pillar cells, inner phalangeal cells, Deiter cells, Hensen cells, Boettcher cells, and/or Claudius cells.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical compositions.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, /toluenesulfonic acid, trifluoroacetic acid, undecylenic acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases used in certain embodiments include isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

### EXAMPLES

### Example 1

This example describes material and methods used in Examples 2-5 bellow.

### Mice

The following mouse strains were used: Fgfr3-iCre (Cox, Liu et al. 2012); Atoh1-GFP (Lumpkin, Collisson et al. 2003), CA-ErbB2 (Xie, Chow et al. 1999), ROSA-floxed-rtTA/GFP (Belteki, Haigh et al. 2005), ROSA-floxed-tdTOMATO (Ail4, (Madisen, Zwingman et al. 2010), and Sox2-CreER (Suh, Consiglio et al. 2007) were all purchased from Jackson Laboratories. Both male and female mice were used equally throughout these experiments. The day that pups were found was designated P0. Institutional Committees on Animal Resources approved all mouse experiments. Genotyping primers and protocols are available upon request.

### Administration of Substances to Mice

Substances include: doxycycline food (200 mg/kg of chow, BioServ S3888); doxycycline hyclate (dox: 100 mg/kg body weight injected using freshly prepared 10 mg/ml in 0.9% sterile saline, Sigma Aldrich D9891); 5-ethynyl-2′-deoxyuridine (EdU: 0.01 mg/kg, injected using a 10 mM EdU stock solution that was dissolved in DMSO and diluted to 40% strength in 0.9% sterile saline, Invitrogen A10044); tamoxifen (0.015 cc/kg injected from a 5 mg/kg in corn oil, both from Sigma, T5648 and C8267). Pups were injected using Ultrafine insulin syringes (Becton-Dickinson 31G 08290-328468).

### Antibodies

The following antibodies were used: ERBB2 (Neu C-18, Santa Cruz Biotechnology SC284); phosphor-ERBB2 (P-Neu Try1248, Santa Cruz SC12352); phosphor-PI3K (P-PI3-Kinase P85α, Santa Cruz SC12929); β-ACTIN (BA3R, ThermoFisher Scientific MA5-15739); SOX2 (Y-17, Santa Cruz SC17320); MYO7A (H-60, Santa Cruz SC25834); JAG1 (C-20, Santa Cruz SC6011); GFP (Abcam ab13970); RFP (Rockland 600-401-379); OCM (N-19, Santa Cruz SC7446); PVALB (EMD Millipore MAB1572). Secondary antibodies were purchased from Jackson Immunoresearch.

### Western Blotting

To obtain fibrocytes, P3 mouse brains were minced in DMEM Glutamax (Gibco), trypsinized (0.25% trypsin/EDTA, Gibco) for 3 minutes at 37° C., neutralized with 10% FBS (Hyclone SH30088) in DMEM, triturated, filtered through a 40 μm nylon mesh, and plated on uncoated plates in DMEM Glutamax, with 10% FBS, 1% pen/strep and 25 mM HEPES, and fed every 2 days. After reaching confluence (around 6-7 days), cells were re-plated in 6-well plates at 10⁶ cells/well. To assay adenovirus activity, wild-type fibrocytes were infected for 24 hours and then extracted in RIPA buffer supplemented with HALT protease and phosphatase inhibitors (Thermo Scientific, 78430 & 78420, respectively). To assay transgene activity, transgenic fibrocytes were stimulated with freshly prepared 2 μg/ml dox (Fisher BP2653) prior to extraction. Extracts were sonicated and quantified (Micro BCA Protein Assay Kit, Thermo, 23235). 20 μg of protein per lane were boiled with Laemmli buffer, subjected to PAGE (12% Mini PROTEAN Gels, BioRad, 4561043), transferred to a nitrocellulose membrane (Sigma, GE 10600016), and probed with primary antibodies (1:1000) in TBST with 5% nonfat milk overnight at 4° C. Secondary antibodies conjugated with horseradish peroxidase were further incubated with the blot in the same buffer for 1 hour at 20° C. Signal was revealed with SuperSignal West Pico Chemiluminescence Substrate (Thermo Scientific, 34087) and X-ray film (Kodak BioMax).

### Tissue Processing

For sectioning, P8 and P14 mice were euthanized with carbon dioxide and decapitated, and P2-P3 pups were decapitated. Inner ears were fixed at least overnight in 4% paraformaldehyde at 4° C. P8 and P14 inner ears were decalcified for 3 days in 100 mM EDTA. P14 inner ears were cryoprotected in 30% sucrose, embedded in OCT, frozen in liquid nitrogen, and sectioned at 20 microns. Sections were dried at 50° C., washed in PBS, blocked with 5% donkey serum in PBS with 0.5% triton for 1 hour, and incubated overnight in block containing primary antibodies diluted to 1:500. After washing, sections were incubated for 2 hours in secondary antibodies diluted to 1:500 at 20° C. Sections were mounted in PROLONG GOLD (Invitrogen P36930). Cultures were also fixed in fresh 4% paraformaldehyde and stained with similar protocols. EdU reactions (Invitrogen C10339 or C10340) were performed prior to antibody staining according to the manufacturer's instructions. For whole mount, P2-P3 cochlear organs were dissected and immersed in fresh 4% paraformaldehyde in PBS. P8 and P14 cochleae, fixed and decalcified as described above, were dissected into three large pieces (Montgomery and Cox 2016). For immunostaining, whole mount tissue was processed similarly to sections, although P8 and P14 cochleae were additionally boiled in 10 mM citric acid (pH 6.0) for 15 minutes after the EdU reaction to facilitate staining.

### Construction of the ErbB2 Adenoviruses

Plasmid 16259 (human HER2, V654E) and plasmid 16258 (human HER2, K753M) were obtained from Addgene and sequenced. Both plasmids were originally in the vector pcDNA3 with 5′ and 3′ HindIII cloning sites. They were cloned into the HindIII site of pAdTrack-CMV (He, Zhou et al. 1998). These constructs were recombined with pAdEasy in BJ5183 competent cells (Agilent Technologies). Subsequently, Ad5 was produced and titrated using standard methods. Western blot experiments were used to verify the activity of the constructs, as described in the Results section. The GFP virus was purchased from Vector Biolabs. All viral protocols were reviewed by the URMC Biosafety committee to ensure the safety of staff and the environment.

### Cochlear Culture and Infection

P1-P2 pups were decapitated and their cochleae dissected into DMEM/F12 media (Gibco 11330-032) buffered with 15 mM HEPES (Gibco, 15630-080). Cochleae were cultured on Lab-Tek CC2 chamber slides (Nunc 154917) coated with 0.5 mg/ml poly-D-lysine (Sigma P6407) and 2 μg bovine fibronectin (Sigma F1141). After removal of the tectorial membrane, the apical and basal turns were cut away to obtain the middle turn. These were placed in the slide wells with a minimal amount of media and incubated for 10 minutes at 37° C. in 5% CO₂ to facilitate attachment. Middle turns were cultured overnight in DMEM/F12 with 15 mM HEPES, 1 mg/ml penicillin G (Sigma P3032), 2% B27 supplement (Gibco 17504-044), 25 ng/ml EGF (Sigma E1257) and 1% FBS (HyClone SH30088). The next day, the media was replaced with similar culture media, except that it now lacked FBS and contained 2 μM EdU (Invitrogen A10044). Note that in other studies, higher concentrations of EdU have been associated with DNA damage in various stem cell types (Kohlmeier, Maya-Mendoza et al. 2013). In preliminary experiments, it was found that the inclusion of 1% FBS facilitated organ attachment, but its presence during adenovirus infection increased the basal level of SC proliferation. Middle turns were infected with 1-3×10⁷ particles per 500 μl culture in a dedicated virus lab using BSL2+ precautions. For Sox2-Cre lineage tracing, 10 μM (z)-4-hydroxytamoxifen (Sigma H7904) was added on the day of isolation to stimulate Cre activity. All viral procedures were reviewed and approved by the University of Rochester's Institutional Biological Safety Committee.

Cochlear Culture with Pa2g4/EBP1 Inhibitors

Cochleae were isolated from postnatal day 1-day 2 wild type or Atoh1-nGFP mice (Lumpkin, Collisson et al. 2003). The organ of Corti was isolated from the otic capsule, and the nerve tissue and stria vascularis were removed. The organ of Corti was plated on a glass coverslip coated with a 1:10 mixture of Matrigel and DMEM/F12 to promote attachment. Cochlear explants were cultured in a serum-free 1:1 mixture of DMEM and F12, supplemented with Glutamax, N2, and B27. For the treated cochlea, small molecules were added to this culture medium, while the control cochlea was cultured with medium containing DMSO at the same concentration used in the treatments. To measure proliferation, explants were treated with EdU (10 μg/ml) along with the drug or DMSO. Drug-treated explants were cultured for 3 days then fixed in 4% PFA for 30 min.

Confocal Microscopy and Image Analysis

All imaging was done on an Olympus FV1000 laser scanning confocal microscope using the Fluoview software package. ImageJ 64 (NIH) was used to Z-project maximal brightness in confocal stacks. Photoshop (Adobe) was used to set maximal and background levels of projections for the construction of figures.

### Experimental Design and Statistical Analysis

Data fields were blinded and randomized using a deck of cards prior to quantification. Virally infected cochleae were imaged at 20× on a confocal microscope using the stitching function of the FV1000 to obtain a composite of the entire field (3000 px by 3000 px). With only the supporting cell marker channel visible, one individual positioned 200 μm rectangles along the supporting cell region (usually 5-7 per middle turn) and then used the channel as a mask to reveal EdU+ cells. The rectangles were exported as TIFs and renamed using a deck of cards. Another individual blinded to condition counted the EdU+ cells. After unblinding, the rectangles were averaged to obtain a biological replicate and the average of these replicates is presented in the text.

For EdU+ and Myo7+ cells in P8 and P14 confocal stacks, an individual blinded to genotype counted EdU+ nuclei and supernumerary MYO7+ cells in P8 and P14 Fgfr3-iCre/CA-ErbB2 image files by examining stack side views. ANOVA was used to establish statistical significance for data groups and a Student's two-tailed t-test with Bonferroni correction was used to establish pair-wise significance.

To quantify WS3 and WS6-treated cochleae, the length of the sensory epithelium was measured using ImageJ software with the overall length determined from the hook to the apex in each sample. The number of myosin VIIa-positive cells or Edu positive cells in the supporting cell region were manually counted. The total number of cells was counted in each of four cochlear segments of 1200-1400 μm (apical, mid-apical, mid-basal, and basal), density (cells per 100 μm) was then calculated for each segment. Statistical analyses were performed using Prism version 7.0 software; comparisons among groups were made by one-way ANOVA followed by Dunnett's multiple-comparisons test for comparing the mean of each group with the mean of a control group.

### Example 2 In Vitro, CA-ERBB2 Drives Cochlear SC Proliferation in a Non-Cell Autonomous Manner that Correlates with Transient Downregulation of SOX2

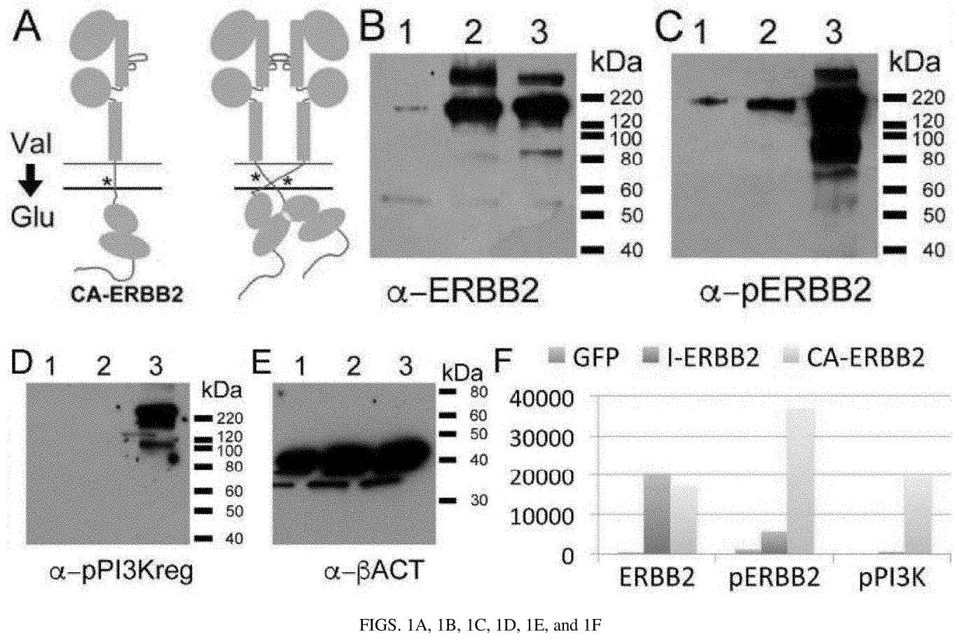

To determine if SCs with active ERBB2 signaling proliferate, adenoviruses (Ad5) were constructed to drive expression of mutated ERBB2 in conjunction with GFP. In many human cancers, mutated HER2/ERBB2 harbors a charged glutamic acid residue in place of a hydrophobic valine located in the transmembrane region (FIG. 1A, asterisk). This glutamic acid facilitates the dimerization of mutated ERBB2 polypeptides, enabling phosphorylation of intracellular tyrosine residues (Stern, Kamps et al. 1988, Weiner, Kokai et al. 1989) and activation of the downstream effector PI3K via sub-unit phosphorylation (p185/PI3Kr). In previous studies regarding SC proliferation after dissociation, inhibitors of PI3K blocked BrdU incorporation in a dose-dependent fashion (White, Stone et al. 2012). Inventors used two ErbB2 constructs derived from human cancer studies (Li, Pan et al. 2004): CA-ErbB2, containing this activating mutation, and I-ErbB2, in which the valine is instead mutated to isoleucine; this mutation fails to drive auto-phosphorylation. Inventors tested both constructs, along with a GFP-only control, in fibrocyte culture by Western blot (FIGS. 1B-E). While ERBB2 immunoreactivity was detected in culture extracts infected with either virus (FIG. 1B, α-ERBB2), downstream events such as phosphorylation of ERBB2 or the PI3K regulatory unit were only observed consequent to CA-ErbB2 infection (FIG. 1C, D, α-pERBB2 and α-pPI3Kreg). Semi-quantitative analysis of the blots illustrates the profound differences between the signals (FIG. 1F).

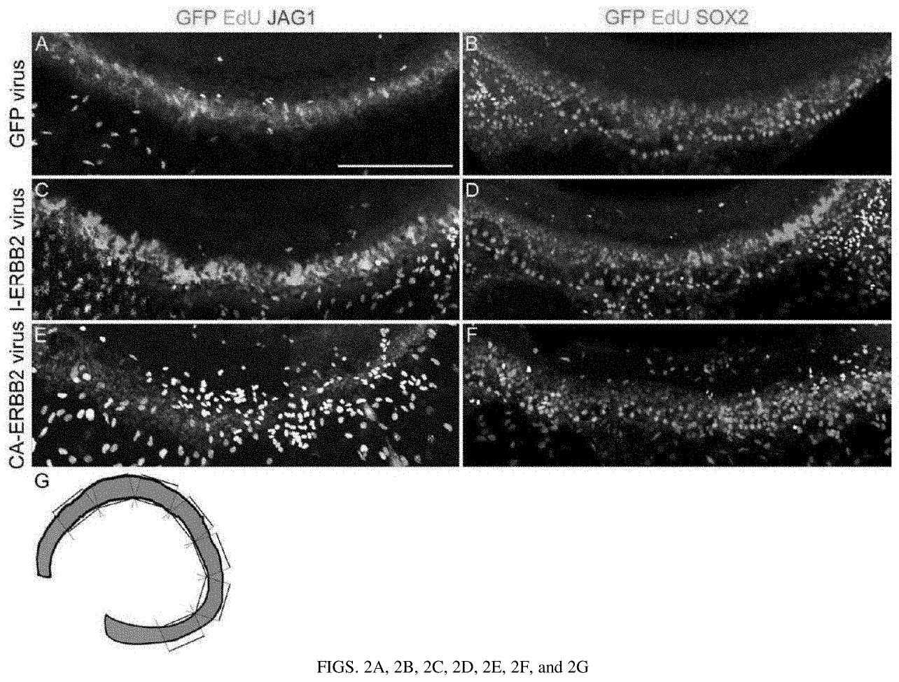

Inventors infected cultures of neonatal cochlear middle turns with the three adenoviruses. Twenty-four hours later, proliferation was assayed by EdU incorporation (FIG. 2, white). In previous experiments, 24 hours was sufficient for dissociated SCs to re-enter the cell cycle but not long enough for them to complete more than one cycle (White, Stone et al. 2012). Consequently, counting cells at 24 hours does not over-estimate the occurrence of cell cycle re-entry. Infected cells expressed GFP (FIG. 2, green), and anti-JAG1 (FIG. 2A, C, E, red) was used to label SCs. EdU incorporation was focal, and it was not evenly distributed through the organ (FIG. 2E). To avoid field selection bias, inventors quantified proliferation in these middle turn cultures from stitched confocal images, positioning 200 μm long rectangles on these images with only the JAG1 channel visible, and blinding and randomizing the resulting images of EdU+ cells revealed through the JAG1 mask (FIG. 2G). Increased proliferation was observed in CA-ErbB2-infected cultures. The overall results were significant (p=0.04, ANOVA, n=24 total organs). Proliferation within the JAG1+ SC region did not differ between GFP-infected and I-ErbB2 infected cultures (39.5±9.2 EdU+ cells/mm vs. 37.6±9.9 EdU+ cells/mm, p=0.89, two-tailed t-test, n=6-8 organs per condition). However, a significant increase in EdU incorporation was observed in the SC region marked by anti-JAG1 after CA-ErbB2 infection compared to GFP only (72.9±11.2 EdU+ cells/mm, p=0.04, two-tailed t-test, n=10 organs for CA-ERBB2 and 8 organs for GFP).

Since virally infected cells express the lineage tracer GFP, images of these cultures were examined to determine if infected cells proliferate (FIG. 2E). Surprisingly, EdU incorporation (FIG. 2E, red) was observed in the SC region in the cells adjacent to the CA-ErbB2 infected cells (FIG. 2E, green), indicating a non-cell autonomous effect. These experiments were replicated using anti-SOX2 to mark the SCs (FIGS. 2B, D, and F, cyan). Remarkably, co-localization of EdU with SOX2 was rarely observed in cultures infected with CA-ErbB2 (FIG. 2F, cf. white and cyan).

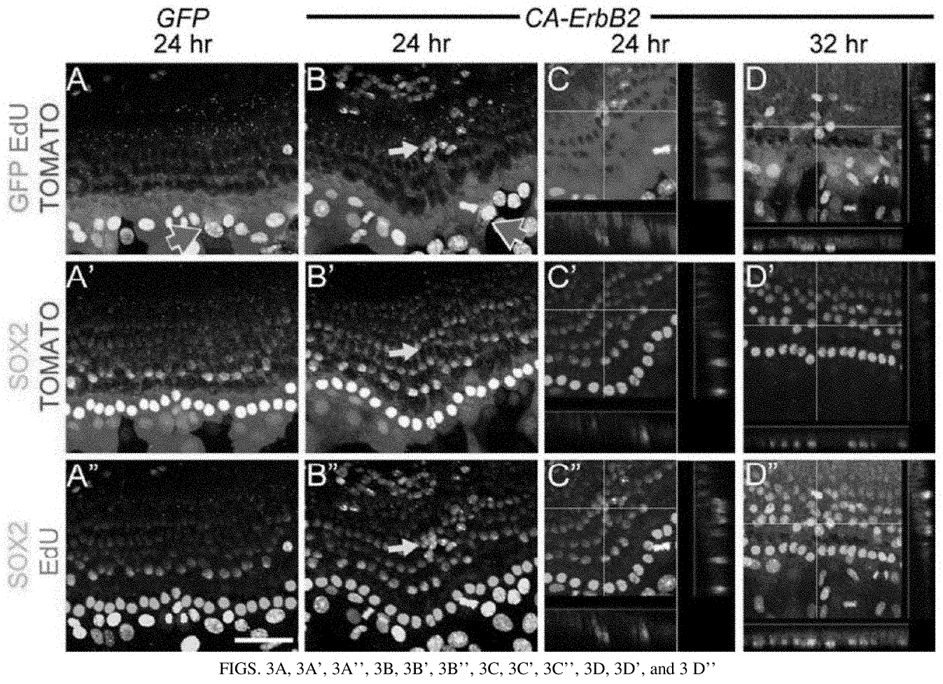

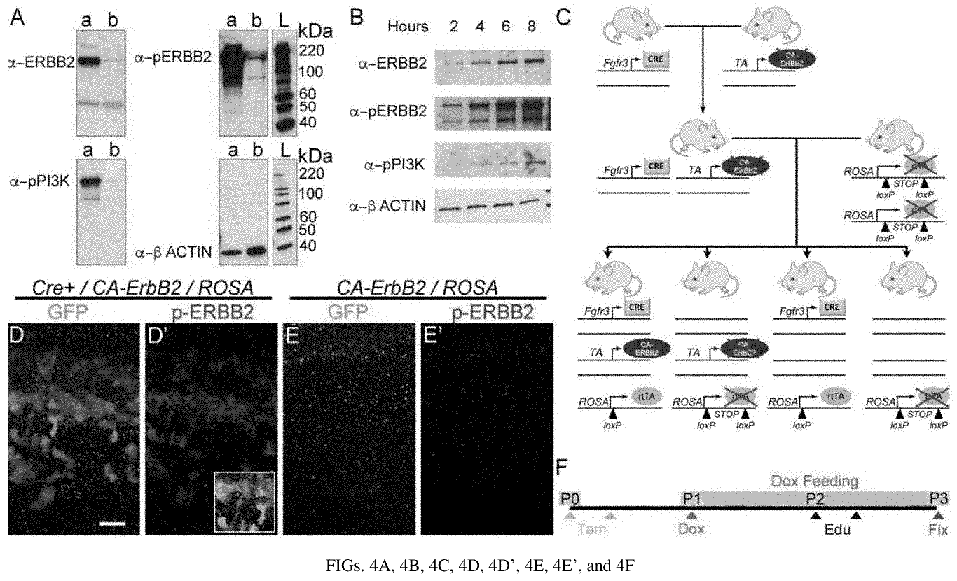

With the lack of co-localization between EdU+ and SOX2+ cells, it was wondered if SOX2 protein is downregulated in SCs when they begin mitosis. Previous studies have implicated SOX2 in the regulation of Cdkn1b/p27Kip1 in cochlear SCs (Liu, Walters et al. 2012). In order to determine if these in vitro proliferating cells were originally SOX2+, inventors crossed a Sox2-CreER knock-in line to a ROSA-floxed tdTomato line and used the progeny for infection experiments. Td-TOMATO expression was induced with 4-hydroxytamoxifen in culture prior to viral infection (FIG. 3). Cultures were assayed for infection, marked by GFP (green staining, pink arrows), Sox2-lineage TOMATO expression (red staining), SOX2 protein expression (cyan staining), and EdU incorporation (white staining, yellow arrows). Little proliferation was observed in the GFP-infected cochleae at 24 hours (FIG. 3A, white). Consistent with previous experiments, inventors observed non-cell autonomous proliferation from CA-ERBB2 infection; e.g., EdU+ cells are GFP-negative (FIG. 3C, D, cf. white staining/yellow arrows with green staining/pink arrows). 79.4%±4.6% of TOM+/EdU+ cells did not express SOX2 protein (FIG. 3C cf. 3C′, yellow arrows, cf. white and cyan, n=10). These cells are in the TOMATO+ region (FIG. 3C, cf. white nuclei with red cell bodies). EdU+/SOX2− nuclei were interspersed in the SOX2+ nuclear layer at 32 hours after infection (FIG. 4D). These data are consistent with a downregulation of SOX2 as proliferation was initiated in cochlear SCs.

### Example 3 Constitutively Activated ERBB2 does not Promote Cochlear SC Proliferation In Vivo

To determine if ERBB2 activation could drive proliferation in cochlear SCs in vivo, a Tet-On system was employed to drive a CA-ErbB2 transgene encoding a constitutively active rat ERBB2 protein, which harbors the same valine to glutamic acid mutation used previously (Xie, Chow et al. 1999). Inventors validated the CA-ErbB2 transgene in protein extracts from cultures of fibrocytes derived from mice harboring both a CA-ErbB2 transgene and a functional ROSA-rtTA knock-in gene (FIG. 4A, lanes a), comparing them to extracts isolated from fibrocyte cultures with ROSA-rtTA alone (FIG. 4A, lanes b). Twenty-four hours after doxycycline (dox) addition, ERBB2 protein was evident on Western blots (FIG. 4A, α-ERBB2, cf. lanes a and b). Both ERBB2 and the regulatory unit of PI3K were phosphorylated (FIG. 4A, α-pERBB2, α-pPI3K, cf. lanes a and b). Probing with anti-β-ACTIN revealed similar protein amounts in both extracts (FIG. 4A, α-β-ACTIN, cf. lanes a and b). To determine the onset of PI3K phosphorylation, sister cultures from CA-ErbB2/ROSA-rtTA derived fibrocytes were harvested at 2, 4, 6 and 8 hours after dox addition. Phosphorylation of the PI3K regulatory unit was evident at 8 hours (FIG. 4B). These data indicate that expression of the CA-ErbB2 transgene indeed resulted in phosphorylation of ERBB2 and its downstream target, PI3K.

To express CA-ERBB2 in the cochlear SCs, the Fgfr3-iCre knock-in was used to activate a floxed ROSA-rtTA/GFP knock-in gene in SCs in neonatal CA-ErbbB2 mice (FIG. 4C, (Cox, Liu et al. 2012)). All mice shown for these experiments harbor the floxed ROSA-rtTA-GFP knock-in gene. The injection schedule for inducing CA-ERBB2 and labeling proliferating cells is shown in FIG. 4F. Cochleae from pups sacrificed at P3 were analyzed for p-ERBB2 immunoreactivity (FIG. 4D, E, red) and GFP expression (FIG. 4D, E, green). Co-localization of GFP and p-ERBB2 was readily apparent in animals harboring both Sox2-CreERT and CA-ErbB2 (note FIG. 4D′, inset). At this time point, it was found that in Sox2-CreERT triple transgenic mice, 96% of GFP+ cells also expressed p-ERBB2, and 98% of p-ERBB2+ cells also expressed GFP (n=281). Similarly, in Fgfr3-iCre triple transgenic mice 96% of GFP+ cells also express p-ERBB2, and 89% of p-ERBB2+ cells also express GFP (n=172).

Since SOX2 protein was not detected in most supporting cells as they re-entered the cell cycle, SOX2 protein was examined by immunofluorescence in mice that harbored either Sox2-CreERT or Fgfr3-iCre in addition to Ca-ErbB2 and ROSA-flox-rtTA-GFP modifications. Exposure-matched images of p-ERBB2 (FIG. 4G-I, red) and SOX2 protein (FIG. 4G-I, cyan) show an apparent reduction in the numbers of SOX2+ cells when compared to CA-ErbB2/ROSA-flox-rtTA-GFP mice alone. SOX2+ cells were quantified in blinded confocal stacks from each of these three genotypes. It was found that organ of Corti from CA-ErbB2/ROSA-flox-rtTA-GFP mice contained an average of 212±62 SOX2+ cells/200 μm segment (average±s.e.m.). In Fgfr3-iCre/CA-ErbB2/ROSA-flox-rtTA-GFP and Sox2-CreERT/CA-ErbB2/ROSA-flox-rtTA-GFP mice, 49±1.5 and 74±9.4 SOX2+ cells/200 μm segment were respectively present and significantly different (p=0.013, ANOVA, n=6 fields per genotype from 2-3 cochleae).

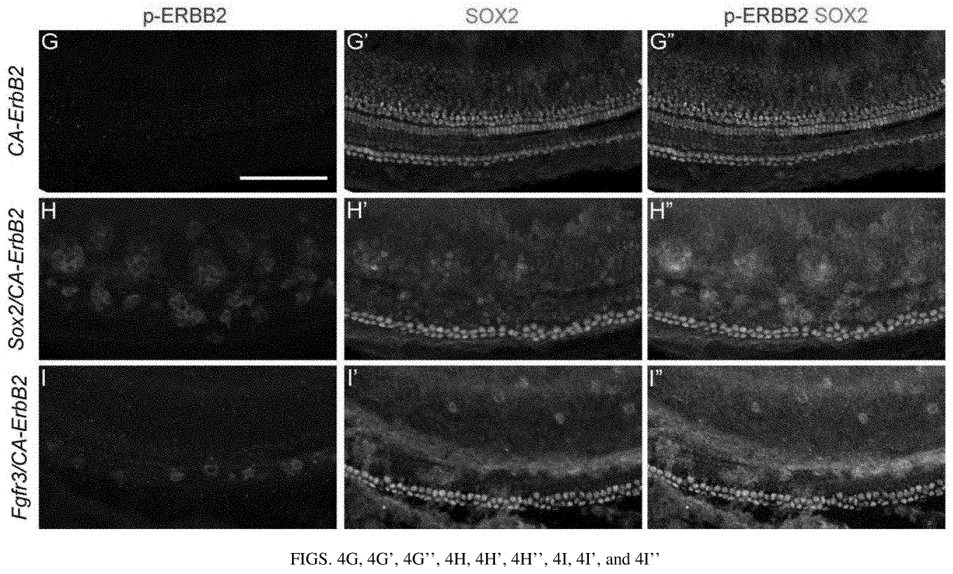

Using the treatment schedule illustrated in FIG. 4F, inventors tested if SCs proliferated in vivo after CA-ERBB2 induction (FIG. 5). Cochleae from each of the three genotypes were co-labeled for SOX2 (FIG. 5A-C, cyan), EdU (FIG. 5A-C, white) and p-ERBB2 (FIG. 5A-C, red). EdU+ nuclei were clustered in and near cells containing phosphorylated ERBB2 (FIG. 5B, C, cf. red and white). The numbers of EdU+ cells were assessed at P8 and P14, using blinded confocal stacks (FIG. 5D) labeled for DAPI (blue), GFP (green), EdU (red) and MYO7 (white). No significant differences were seen in the numbers of EdU+ cells at either P8 or P14 (FIG. 5E), indicating that the activation of CA-ERBB2 is not sufficient to drive significant increases in proliferation in vivo.

Although approximately 1 in 4 pups analyzed at P3 or P8 harbored both Fgfr3-iCre and CA-ErbB2, only 3 in 34 mice were obtained at P14. Similar experiments performed with Sox2-CreERT mice yielded no mice with both a Cre gene and CA-ErbB2 (out of 39 generated, data not shown). Moreover, surviving Fgfr3-iCre+/CA-ErbB2+ mice were small, sickly, and hairless, with wrinkled skin (not shown).

### Example 4 Supernumerary MYO7+ Cells In Vivo Consequent to ERBB2 Activation

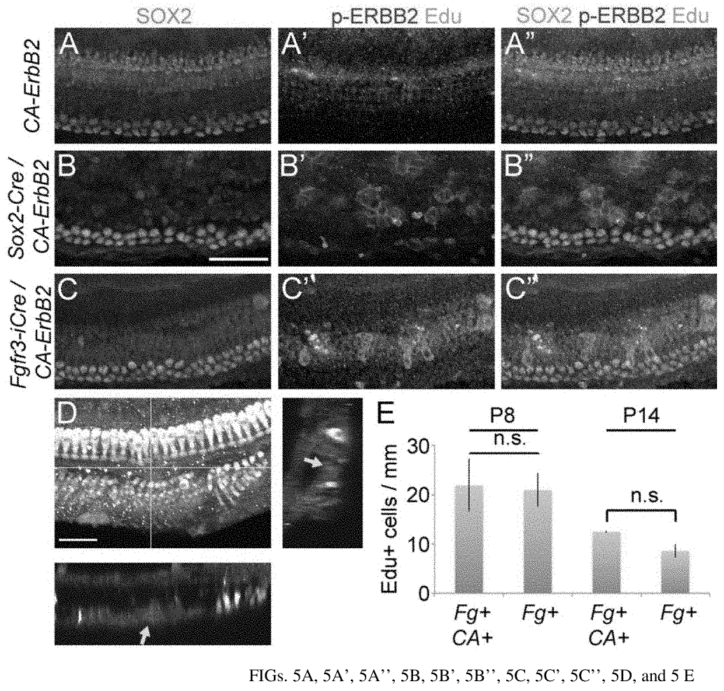

In analyses of P8 and P14 mice, it was surprising to discover many supernumerary MYO7+ cells located in the SC region (FIG. 6). Supernumerary MYO7+ cells were quantified on blinded confocal stacks (FIG. 6A, B). The frequency of supernumerary MYO7+ cells near OHCs ranged from 10 to 30 cells/mm of cochlea, to a maximum of 117 total new MYO7+ cells in one organ (FIG. 6C). Few supernumerary cells were observed in control animals (FIG. 6C) and the increase was statistically significant (p=0.02, student's two-tailed t-test, P8, n=4 per genotype). Mice lacking a CA-ErbB2 transgene had normal complements of HCs, identified with antibodies against MYO7a and Oncomodulin (OCM), juxtaposed to GFP-containing CRE+ cells (FIG. 6D, E). Normal organization was observed in both the mid-base (FIG. 6D) and apical regions (FIG. 6E). Animals harboring a CA-ErbB2 transgene, in contrast, had supernumerary MYO7+ cells that co-expressed OCM, suggesting OHC differentiation (FIG. 6F, G yellow arrows). Such cells were present in the mid-base turn (FIG. 6F, arrows) and in apical regions (FIG. 6G, arrow). Occasionally, supernumerary MYO7+ cells near IHCs were also observed in the apical regions (FIG. 6H, arrow). Supernumerary MYO7+ cells also expressed Parvalbumin (PVALB) (FIG. 6H, cyan). They did not express SOX2 (FIG. 6I, cyan, arrow). They were typically located near GFP+ cells (FIG. 6F, G, cf. red with green) but did not express GFP. These data suggest that ERBB2 signaling is upstream of a short-range paracrine signal that drives the initial events of HC differentiation in young animals in vivo.

### Example 5 Small Molecules that Activate ErbB2 Pathway Promote SC Expansion and Supernumerary MYO7+ Cell Generation

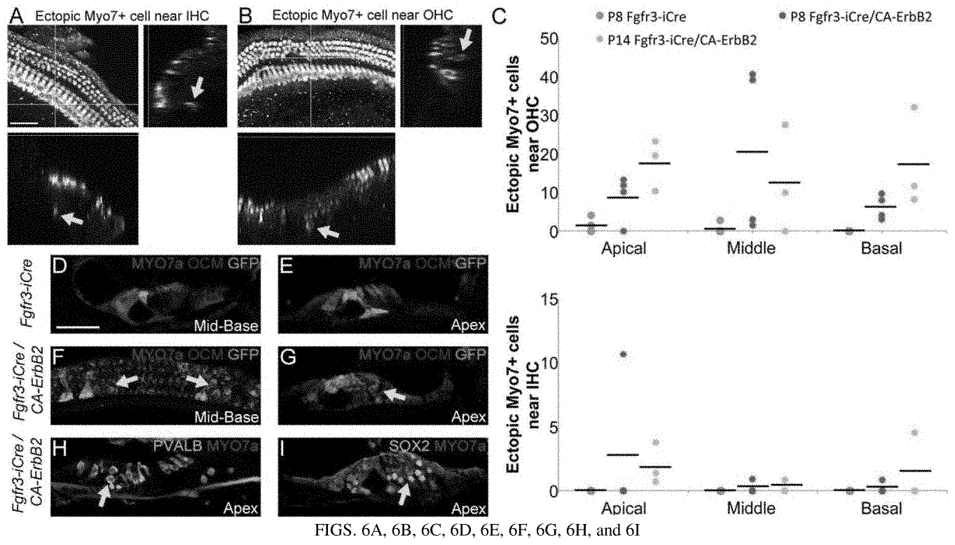

Our findings with the conditional ErbB2 activation animal models have shown that the constitutive activation of ERBB2 can result in SC expansion and ectopic MYO7+ cell generation. To further validate the crucial role of ERBB2 activation, inventors turned to small molecules to pharmacologically activate the ErbB pathway and determine their effect on SC expansion and HC differentiation. Two compounds, WS3 and WS6, were chosen, as these two analogues promoted cell proliferation in growth arrested cells such as islet 3 cells and retinal pigment epithelial (RPE) cells (Shen, Tremblay et al. 2013, Swoboda, Elliott et al. 2013). Through their action on ERBB3 binding protein 1 (EBP1/PA2G4), a component of the ErbB signaling pathway, these compounds reduced the antiproliferative role of PA2G4 and upregulated several cell cycle-activated genes (Squatrito, Mancino et al. 2004, Shen, Tremblay et al. 2013, Swoboda, Elliott et al. 2013). To test the effect of these drugs on SCs, they were applied to cochlear explants derived from Atoh1-nGFP reporter mice (Lumpkin, Collisson et al. 2003). With WS3 or WS6 treatment, additional Atoh1-nGFP-positive cells near OHCs were seen (FIGS. 7A and B) with a gradient from apex to base. The number of MYO7+ cells in the apex was increased by 20 cells/100 μm (FIGS. 7A and 7B).

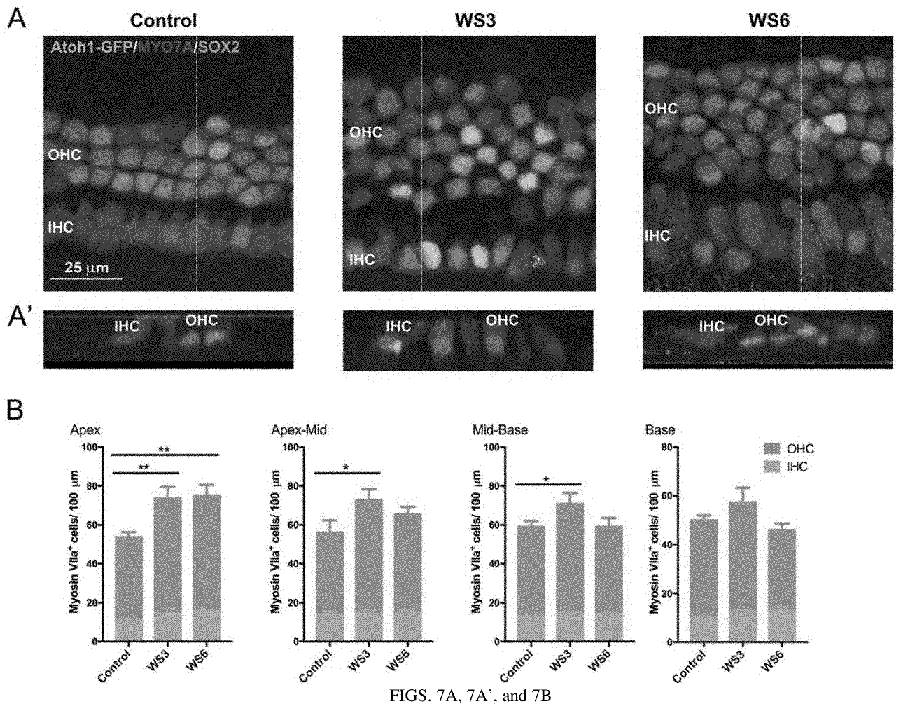

To determine if the drug treatments promoted SC proliferation, the compound were applied to cochlear explants from wild type mice at P1-P2 with EdU present to label the proliferating cells. A large number of EdU+ cells were observed in the SOX2+ SC region in both WS3 and WS6-treated cochleae, with a gradient from apex to base (FIGS. 8A and B). These results were consistent with the enhanced SC proliferation observed with the CA-ErbB2 viral transduction in vitro (FIG. 2). To confirm that the effect of the drugs was mediated through ERBB2 activation, the ERBB2 phosphorylation was analyzed using the protein lysates from cochlear explant. Due to the limited protein quantity from the primary tissue, inventors were not able to detect the ERBB2 protein signal by western blot (data not shown); in breast cancer cell line MCF-7, however, p-ERBB2 level was elevated in response to drug treatment (FIG. 8C), indicating that the ErbB2 signaling pathway was activated by WS3 or WS6. The data suggest that pharmacological activation of ErbB signaling by small molecules promoted SC proliferation and increased MYO7+ cell generation in vitro, similar to what had been observed in the transgenic animal model in vivo and further validating the crucial role of ErbB activation for the inner ear regeneration.

### Example 6 ERBB2 Function in Noise-Damaged Adult Cochleae In Vivo

Gene Expression.

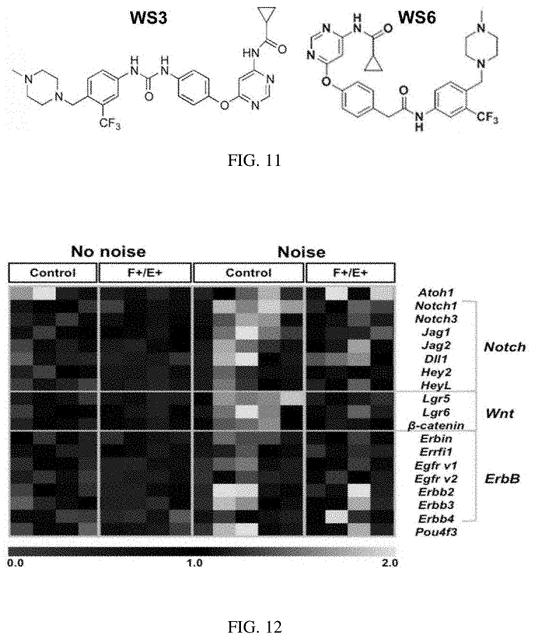

To determine whether noise exposure and CA-ERBB2 expression can influence RNA expression profiles, cochlear RNA was harvested from adult mice harboring the appropriate transgenes and performed qPCR array analysis. Four groups of mice were analyzed: CA-ERBB2 (F+/E+) and control genotype (F+/− and E+/−) mice were compared in both the noise vs. no noise conditions. qPCR results revealed fold differences (FD) in three major pathways: Notch (Notch1, Notch3, Jag1, Jag2, Dll1, Hey2, HeyL), Wnt (Lgr5, Lgr6, β-catenin), ErbB (Erbin, Errfi1, EgfrV1, EgfrV2, ErbB2, ErbB3, ErbB4); and two hair cell specific genes (Atoh1, Brn3.1). Gene expression data was normalized to no-noise control animals (Fgfr3-iCre+/− and CA-ERBB2+/−). In the no noise condition, most of the pathways were down-regulated (FD 0-1) in CA-ERBB2 activated (F+/E+) animals compared to control animals (FIG. 12). After the noise exposure, a mixed pattern as specific pathways became up-regulated (FD 1-2) in F+/E+ animals. Gene up-regulation was also observed in control animals, suggesting that some alterations were due to noise insult alone (FIG. 12).

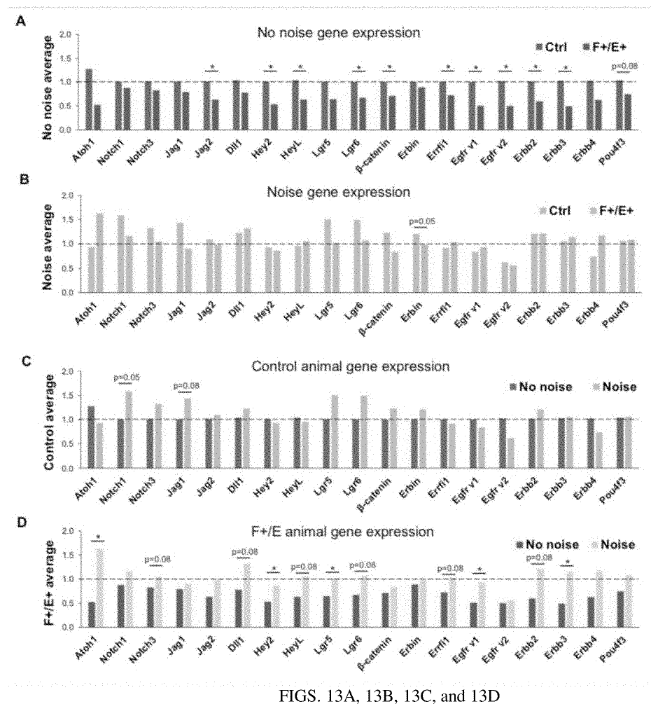

The average expression of each gene was compared in four different categories (FIGS. 13A-D). CA-ERBB2 alone under normal condition significantly down-regulated most genes (FIG. 13A Control vs. F+/E+), which is the same as shown in heat map (FD 0-1). When compared between normal and noise exposed conditions, no significant change was observed in control animals (FIG. 13C, No noise vs. Noise). However, CA-ERBB2 significantly up-regulated Atoh1 (HC specification marker), Hey2 (Notch pathway), Lgr5 (Wnt pathway), Egfr, and ErbB3 (ErbB pathway) (FIG. 13D No noise vs. Noise), instead of repressing these pathways under normal condition (FIG. 13A). In summary, these data indicated that CA-ERBB2 inhibits the transcription of many regenerative genes without noise but appears to stimulate expression of HC regeneration gene and other ErbB family genes after noise damage.

Partial Functional Recovery from Noise Damage 2-3 Months after ERBB2 Activation.

To determine whether transient ERBB2 activation alters long-term hearing, inventors drove expression of CA-ERBB2 in 1 M old mice and harvested their inner ears 2-3 months later. The experimental timeline is described in FIG. 14A. ABR/DPOAE was measured at the beginning of the experiment, again 3 days post Tam (DPT), and again 1 and 2 months post Tam (control n=6, F+/E+ n=4). A transient ABR dB shift was observed at 3 DPT due to Tamoxifen injection but prior to CA-ERBB2 activation. No differences were observed in ABR or DPOAE thresholds between control and CA-ERBB2 activated (F+/E+) mice at 30 DPT or 60 DPT (FIGS. 14B-E), indicating that CA-ERBB2 treatment alone does not affect normal hearing in long-term.

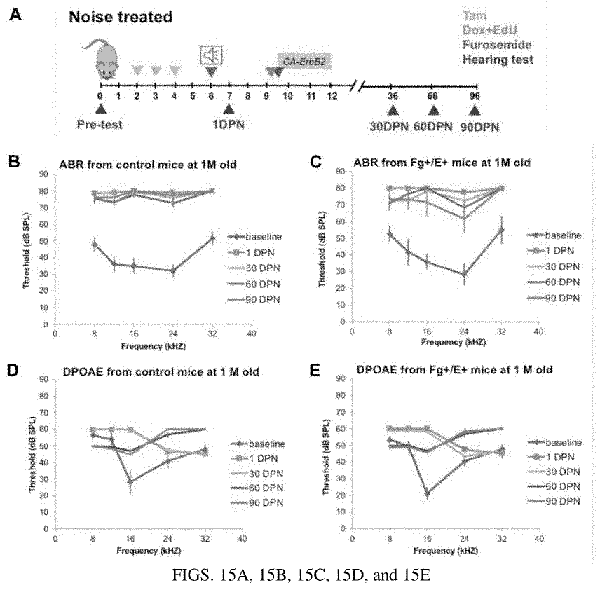

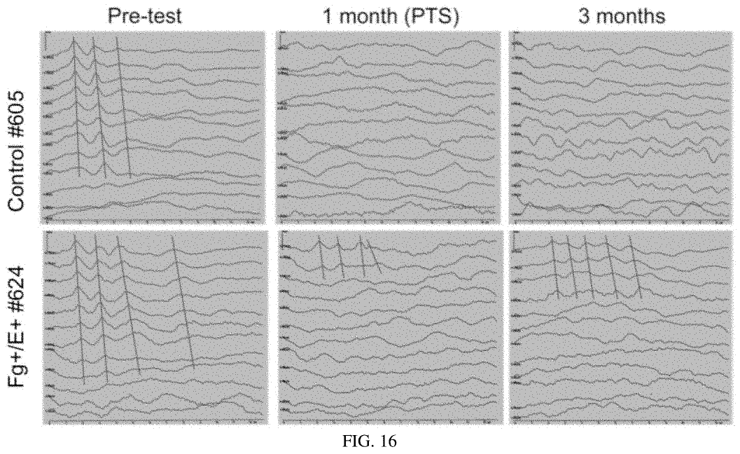

To evaluate if transient CA-ERBB2 expression promotes hearing recovery after noise damage, 1 M old mice were exposed to noise (8-16 kHz band) at 110 dB for 2 hours (FIG. 15). CA-ERBB2 was activated at 3-day post noise (DPN) (FIG. 15A, control genotypes n=10, F+/E+ n=6). 1 M old mice exposed to the same noise exhibited severe damage, indicated by non-reversible ABR threshold elevation (FIG. 15B). This is consistent with a previously reported sensitivity to noise damage in young adult mice (Ohlemiller K K, et al. Hear Res. 2000; 149(1-2):239-47). Both ABR and DPOAE results showed significant dB SPL threshold elevation at 1, 2 and 3-month post noise for both control and most of the CA-ERBB2 activated mice (FIG. 15B). Strikingly, it was found that one CA-ERBB2 activated mouse was significantly deaf at 1DPN, but its ABR thresholds gradually improved at 12, 16 and 24 kHz over the next 30-90 days. An example from its ABR recording at 24 kHz demonstrated the partial recovery from permanent threshold shift (PTS) compared to the control mouse (FIG. 16). In summary, these results indicate that noise exposure induced severe hearing impairment in 1 M old young adult mice, and CA-ERBB2 promoted partial hearing recovery at 2-3 months post noise.

An extended time frame was used to evaluate the effects of activating ERBB signaling in mice, in part because birds require 4-8 weeks to recover their hearing (Ryals B M, et al. Hear Res. 2013; 297:113-20). In addition, when hair cell differentiation can be stimulated in adult mammalian utricles, peak hair cell production occurs over a month after induction (Golub J, et al. Inhibition of Gamma-Secretase Promotes Non-Mitotic Hair Cell Regeneration in the Adult Mouse Utricle. ARO; 2011; Baltimore, Md.; and Lin V, et al., J Neurosci. 2011; 31(43):15329-39). Thus, the test subjects were allowed several months for hearing recovery. The effects observed are changes to the permanent threshold shift mice incur from noise exposure, as opposed to changes in temporary threshold shifts, as they were observed 3 months after noise exposure, in comparison to the threshold shift determined one month after exposure.

### Example 7 ERBB2 Signaling in Supporting Cells Promoted Hearing Recovery in Adults after Noise Damage

It was hypothesized that the activation of ERBB2 signaling in supporting cells could promote hearing recovery in adults after noise damage. To test this hypothesis, an inducible genetic system was used. The system had three parts: (1) a transgene where a constitutively active ERBB2 gene (CA-ERBB2) was controlled by a bacterial promoter, called TA; (2) a “knock-in” gene that would drives expression of the TA protein; and (3) another “knock-in” gene that expresses an inducible CRE DNA recombinase, which can remove the stop codon from the TA gene, enabling its expression.

There are two important aspects to the second “knock-in” gene, which is called “ROSA”. First, it had a “floxed” stop codon at its beginning, so that it needed to be activated to work. Second, the TA used is only functional when an antibiotic, doxycycline (dox) is present. The final gene was under the control of a supporting cell-specific promoter, and further requires an injection of tamoxifen to work. Accordingly, only when all three genes are present in a mouse, and the correct drugs are administered, will CA-ERBB2 be expressed. The supporting cell specific CRE is likely to be expressed in a minority of supporting cells (<10%).

Shown in the table below is the timeline of the experiment. The experiment was done twice. All mice were included except for mice that died before the final time point and mice whose 2^{nd} and 3^{rd} hearing tests showed a reduced change in threshold. The latter condition excludes mice that either started somewhat deaf, or that did not get a sufficient noise damage (sufficient is >30 dB threshold shift on average for all five frequencies tested). Two mice were excluded on that basis. A total of 6 mice with all three genes (CA-ERBB2) were compared to 6 mice with only two genes (control). Two controls had the CRE gene and ROSA, and four controls had the first transgene and ROSA.

The auditory brainstem response or ABR hearing test was carried out at five frequencies where mice can hear: 8, 12, 16, 24, and 32 kHz. Higher values of ABR indicate worse hearing: i.e. sounds must be louder for the mice to detect them.

| | | | | 6 weeks + | 6 weeks + | | | |
|---|---|---|---|---|---|---|---|---|
| Mouse age | 4 weeks | 5 weeks | 6 weeks | 1 day | 3 days | 10 weeks | 14 weeks | 18 weeks |
| Event | CRE | 1^{st} hearing test | Noise | 2^{nd} hearing test | DOX | 3^{rd} hearing test | 4^{th} hearing test | 5^{th} hearing test |
| | activation | (“pre-test”) | damage | (“1 DPN”) | treatment | (“1 MPN”) | (“2 MPN) | and euthanasia |
| | | | | | | | | (“3 MPN”) |

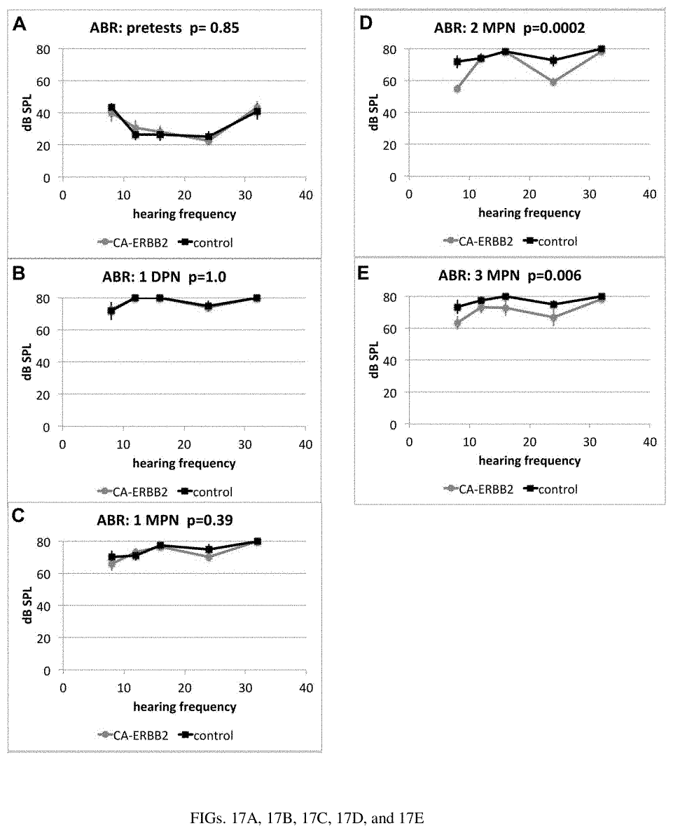

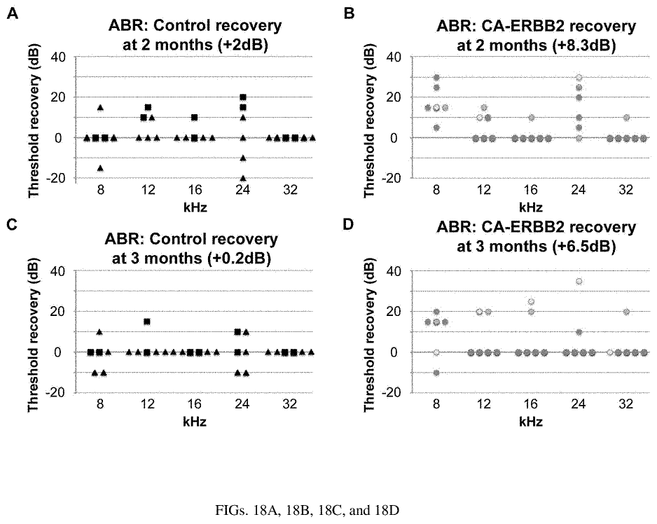

The results were shown in FIG. 17 and FIG. 18. FIG. 17 shows and directly compares the averages of the ABR results from the CA-ERBB2 and control mice. Error bars represent the standard error of the mean. The p values shown in the headlines were calculated using ANOVA. As shown in figure, prior to noise damage, both genotypes had identical hearing. Immediately after noise damage, both genotypes had identical hearing, indicating that both lines damaged similarly. One month after noise damage, both genotypes again had similar hearing. Yet, 2 and 3 months after noise damage, the CA-ERBB2 mice had significantly improved thresholds on average.

To further illustrate the effect, FIG. 18 shows the hearing (threshold) recovery for each mouse as a dot plot. Threshold recovery was calculated by subtracting the threshold at the later date (2 or 3 months) from the threshold immediately after noise exposure, for each mouse at each frequency. Control mice were four Fg-CRE mice (triangles) and 2 ERBB mice (squares). CA-ERBB2 mice are represented with circles. Two CA-ERBB2 mice were highlighted: one is represented by pale circles, and one by medium circles. These mice had the best recovery overall. The lowest frequency (8 kHz) was the best for recovery.

The results indicate that ERBB2 signaling in supporting cells promoted hearing recovery in adults after noise damage.

### Discussion.

The mammalian cochlea lacks the regenerative capacity of non-mammalian counterparts. Here the inventors tested intrinsic ErbB2 signaling as a candidate regulator of mammalian cochlear regeneration. It was found that neonatal mouse SCs expressing a constitutively activated ERBB2 receptor (CA-ERBB2) promote SC proliferation in vitro. Moreover, cochleae with CA-ERBB2 expression developed supernumerary MYO7+ cells in vivo. Both proliferation and MYO7+ induction were observed when small molecule effectors stimulate ERBB3 signaling in vitro. These data suggest ERBB2 signaling as a pathway in regulating the regeneration response.

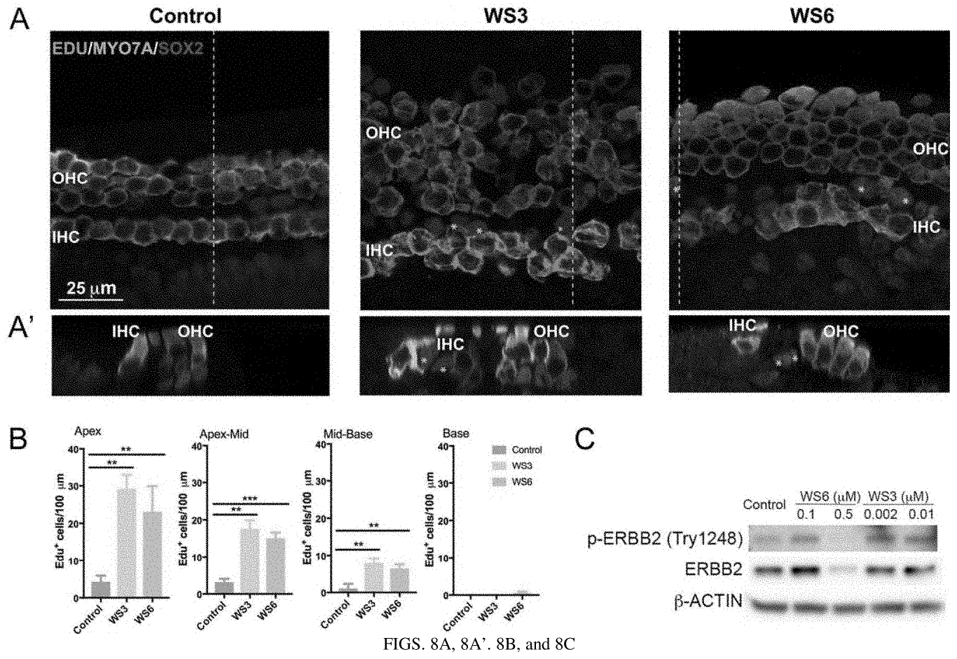

The findings are summarized in FIG. 9. Each method used to modulate ERBB signaling had similar, but not identical results. The use of the virus or transgenic technology to drive CA-ERBB2 activity enabled lineage tracing of transduced cells (dark green). Strikingly, it was found that it is the cells nearby these transduced cells that respond, by either modulating SOX2 expression (cyan), proliferating (red), or inducing MYO7 (white). That ERBB2 signaling is non-cell autonomous implies the existence of downstream signals that regulate these activities, a completely unexpected result. SOX2 modulation was observed with CA-ERBB2 expression both in vitro (FIG. 2, 3) and in vivo (FIG. 4-6), but not after ERBB3 activation with small molecules. Similarly, proliferation was observed with both in vitro systems, but not in vivo. This finding suggests that additional constraints provided by cochlear structure in vivo could play a role in preventing proliferation, for example by limiting cell growth. Finally, the inventors observed significant levels of MYO7 induction in the two systems where it could be assessed. Although not shown here, it was found that infected cochlear explants became too disorganized and spread out to accurately quantify MYO7+ cells within two days of viral transduction (FIG. 9, “Not assayed”). Importantly, supernumerary MYO7+ cells were observed throughout the cochlea (FIG. 6). This rules out the possibility that ERBB2 activation affects secondary processes such as convergent extension. Convergent extension is complete in the basal and middle cochlea by birth (Chen, Johnson et al. 2002), when ERBB2 activation is initiated. In concert, these findings strongly implicate ERBB family signaling in the regulation of cochlear regeneration events.

Each CA-ErbB2 transgene used in this study was derived from carcinoma cells (Xie, Chow et al. 1999, Li, Pan et al. 2004). In many tumors, CA-ERBB2 acts cell autonomously to promote proliferation. Surprisingly, constitutively active ERBB2 signaling in certain non-proliferating tumor cells drives a change in their secretome that promotes neighboring cells to change fate and become metastatic (Angelini, Zacarias Fluck et al. 2013). The data disclosed herein strongly indicate that CA-ERBB2 triggers expression of regenerative signals for responding neighbor cells, which parallels the second mechanism. Recently, others have investigated heart regeneration using this CA-ERBB2 transgene (D'Uva, Aharonov et al. 2015). Transient induction of CA-ERBB2 following myocardial ischemic injury can improve heart function, by the proliferation and de-differentiation of cardiomyocytes via 13-CATENIN accumulation (D'Uva, Aharonov et al. 2015). Findings in both mammalian cochlear and heart regeneration suggest CA-ErbB2 is driving specific regeneration activities rather than oncogenic transformation.

It is reported herein that SOX2 protein expression is reduced in both proliferating and trans-differentiating SCs after ErbB2 transduction (FIGS. 2-6). Related proliferation data is consistent with reports that SOX2 directly activates p27kip1, a cell cycle inhibitor in SCs. Targeted deletion of Sox2 in postmitotic SCs leads to inner pillar cell (a subtype of SCs) proliferation (Liu, Walters et al. 2012). During development, ectopic expression of SOX2 can drive both SC and HC markers (Pan, Jin et al. 2013, Puligilla and Kelley 2017). SOX2 binds to the Atoh1 promoter and increases its expression levels (Neves, Uchikawa et al. 2012, Kempfle, Turban et al. 2016, Puligilla and Kelley 2017). Interestingly, SOX2 also drives expression of ATOH1 repressors, including Hes genes and Id1 (Neves, Uchikawa et al. 2012, Neves, Vachkov et al. 2013). In this so-called incoherent feed-forward loop (Alon 2007), such contradictory effects of the inducer SOX2 drive a pulse-like accumulation of the target, ATOH1. The data disclosed herein support the model that prolonged SOX2 expression may maintain the post-mitotic SC phenotype.

Recent efforts from other laboratories describe potential candidates for CA-ERBB2's as yet unknown downstream signal. Supernumerary HCs are observed when NOTCH1 signaling is reduced in SCs (Lanford, Lan et al. 1999, Yamamoto, Tanigaki et al. 2006, Mizutari, Fujioka et al. 2013). As Notch signaling maintains SOX2 expression in SCs during the neonatal period (Lanford, Lan et al. 1999, Kiernan, Xu et al. 2006, Pan, Jin et al. 2013), this pathway fits well with the data disclosed herein. Stabilization of the Wnt effector 13-CATENIN in neonatal SCs promotes their proliferation (Chai, Kuo et al. 2012, Shi, Hu et al. 2013, Kuo, Baldwin et al. 2015). This treatment also increases Atoh1 expression, likely through direct interactions of 13-CATENIN with the Atoh1 enhancer (Shi, Cheng et al. 2010). SHH treatment also promotes rodent SC proliferation in vitro (Lu, Chen et al. 2013). ERBB2 binds to other ErBB family proteins and various receptor tyrosine kinases (Jones, Gordus et al. 2006). ERBB2 heterodimers with other ERBB family proteins are the active receptors for growth factor ligands and amplify ERBB signaling by slowing endocytosis and decreasing the receptor-recycling period [reviewed in (Bertelsen and Stang 2014)]. This would enhance the existing growth factor signaling in our CA-ERBB2 model. Further experiments can be carried out to determine which of these pathways may act downstream of CA-ERBB2. Further experiments can also be carried out to determine if there is heterogeneity in the responses of SCs to intrinsic CA-ERBB2 signaling, which SCs produce factors to induce regeneration-like responses, if regeneration-like responses have a concentration dependence, and if inhibitor molecules that limit the scope of regeneration are also produced.

In addition to investigating ERBB2 in transgenic models, inventors identified small molecules, such as WS6 and WS3, which exhibited similar regenerative potential by regulating ErbB2 signaling (FIGS. 7 and 8). WS6 treatment increases p3-cell mass in a rodent diabetes model by promoting cell proliferation (Shen, Tremblay et al. 2013), while WS3 expands retinal pigment epithelium (RPE) cells and preserves vision when the cells are transplanted into a retinal degeneration model (Swoboda, Elliott et al. 2013). Here effects of WS6 and WS3 were assessed in cochlear explant culture. Besides direct activation of ERBB2 phosphorylation, these two PA2G4 inhibitors may alter the ERBB signaling cascade through other means. EBP1/PA2G4 is expressed throughout the sensory region of the P0 mouse cochlea (Hertzano and Orvis 2016). PA2G4 negatively regulates ERBB2 mRNA and protein level via transcriptional mechanisms (Ghosh, Awasthi et al. 2013). A recent study showed that PA2G4 interacts with the ERBB downstream molecule PI3K and inhibits its kinase activity (Ko, Kim et al. 2014). A PA2G4 inhibitor might increase PI3K activity and enhance the ERBB signaling. The results described herein provide an approach using drugs to enhance HC regeneration in future studies.

During normal cochlear homeostasis, ErbB signaling is implicated in the regulation of spiral ganglion neuron (SGN) innervation. Expression of a dominant negative ERBB2 variant in SCs shows that this signaling is crucial for the survival of SGNs (Stankovic, Rio et al. 2004). How then, can ERBB signaling be tasked with two completely separate functions: regeneration and innervation? The intracellular signaling sites on ERBB2 are highly promiscuous, strongly interacting with at least 17 distinct protein domains (Jones, Gordus et al. 2006). Mammalian SCs may express different levels of specific ERBB interactants compared to bird or fish SCs. This invention implicates PA2G4, a regulator of ERBB3 signaling, in blocking SC regeneration activities. It is possible that evolution may have redirected ERBB signaling in mammals towards facilitating innervation and away from regeneration.

In summary, inventors demonstrate a potential role of ERBB signaling in stimulating SC proliferation and supernumerary MYO7+ cell differentiation in neonatal mouse cochlea. Using multiple methods to activate ERBB signaling, the inventors found ERBB to be upstream in promoting these processes. Taken together with ERBB2's role in detecting stretch damage in other epithelial tissues, the findings suggest that within some SCs, ERBB signaling initiates a cascade of downstream signaling pathways that enhance regeneration activity.

### REFERENCES

Alon, U. (2007). “Network motifs: theory and experimental approaches.” Nat Rev Genet 8(6): 450-461.
Angelini, P. D., M. F. Zacarias Fluck, K. Pedersen, J. L. Parra-Palau, M. Guiu, C. Bernado Morales, R. Vicario, A. Luque-Garcia, N. P. Navalpotro, J. Giralt, F. Canals, R. R. Gomis, J. Tabernero, J. Baselga, J. Villanueva and J. Arribas (2013). “Constitutive HER2 signaling promotes breast cancer metastasis through cellular senescence.” Cancer Res 73(1): 450-458.
Bainbridge, K. E. and M. I. Wallhagen (2014). “Hearing loss in an aging American population: extent, impact, and management.” Annual review of public health 35: 139-152.
Belteki, G., J. Haigh, N. Kabacs, K. Haigh, K. Sison, F. Costantini, J. Whitsett, S. E. Quaggin and A. Nagy (2005). “Conditional and inducible transgene expression in mice through the combinatorial use of Cre-mediated recombination and tetracycline induction.” Nucleic Acids Res 33(5): e51.
Bertelsen, V. and E. Stang (2014). “The Mysterious Ways of ErbB2/HER2 Trafficking.” Membranes (Basel) 4(3): 424-446.
Brignull, H. R., D. W. Raible and J. S. Stone (2009). “Feathers and fins: non-mammalian models for hair cell regeneration.” Brain Res 1277: 12-23.
Chai, R., B. Kuo, T. Wang, E. J. Liaw, A. Xia, T. A. Jan, Z. Liu, M. M. Taketo, J. S. Oghalai, R. Nusse, J. Zuo and A. G. Cheng (2012). “Wnt signaling induces proliferation of sensory precursors in the postnatal mouse cochlea.” Proc Natl Acad Sci USA 109(21): 8167-8172.
Chardin, S. and R. Romand (1995). “Regeneration and mammalian auditory hair cells.” Science 267: 707-709.
Chen, P., J. E. Johnson, H. Y. Zoghbi and N. Segil (2002). “The role of Math1 in inner ear development: Uncoupling the establishment of the sensory primordium from hair cell fate determination.” Development 129(10): 2495-2505.
Corwin, J. T. and D. A. Cotanche (1988). “Regeneration of sensory hair cells after acoustic trauma.” Science 240(4860): 1772-1774.
Cox, B. C., Z. Liu, M. M. Lagarde and J. Zuo (2012). “Conditional gene expression in the mouse inner ear using Cre-loxP.” J Assoc Res Otolaryngol 13(3): 295-322.
Crowe, S. J., S. R. Guild and L. M. Polvogt (1934). “Observations on the pathology of high-tone deafness.” Bulletin of the Johns Hopkins Hospital 54(5): 315.
D'Uva, G., A. Aharonov, M. Lauriola, D. Kain, Y. Yahalom-Ronen, S. Carvalho, K. Weisinger, E. Bassat, D. Rajchman, O. Yifa, M. Lysenko, T. Konfino, J. Hegesh, O. Brenner, M. Neeman, Y. Yarden, J. Leor, R. Sarig, R. P. Harvey and E. Tzahor (2015). “ERBB2 triggers mammalian heart regeneration by promoting cardiomyocyte dedifferentiation and proliferation.” Nat Cell Biol 17(5): 627-638.
Doetzlhofer, A., P. M. White, J. E. Johnson, N. Segil and A. K. Groves (2004). “In vitro growth and differentiation of mammalian sensory hair cell progenitors: a requirement for EGF and periotic mesenchyme.” Dev Biol 272(2): 432-447.
Ghosh, A., S. Awasthi and A. W. Hamburger (2013). “ErbB3-binding protein EBP1 decreases ErbB2 levels via a transcriptional mechanism.” Oncol Rep 29(3): 1161-1166.
He, T. C., S. Zhou, L. T. da Costa, J. Yu, K. W. Kinzler and B. Vogelstein (1998). “A simplified system for generating recombinant adenoviruses.” Proc Natl Acad Sci USA 95(5): 2509-2514.
Hertzano, R. and J. Orvis. (2016). “gEAR—gene Expression Analysis Resource web portal.” 2017, from http://gear.igs.umaryland.edu/index.html,
Hume, C. R., M. Kirkegaard and E. C. Oesterle (2003). “ErbB expression: the mouse inner ear and maturation of the mitogenic response to heregulin.” J Assoc Res Otolaryngol 4(3): 422-443.
Jones, R. B., A. Gordus, J. A. Krall and G. MacBeath (2006). “A quantitative protein interaction network for the ErbB receptors using protein microarrays.” Nature 439(7073): 168-174.
Kempfle, J. S., J. L. Turban and A. S. Edge (2016). “Sox2 in the differentiation of cochlear progenitor cells.” Sci Rep 6: 23293.
Kiernan, A. E., J. Xu and T. Gridley (2006). “The Notch ligand JAG1 is required for sensory progenitor development in the mammalian inner ear.” PLoS Genet 2(1): e4.
Ko, H. R., C. K. Kim, S. B. Lee, J. Song, K. H. Lee, K. K. Kim, K. W. Park, S. W. Cho and J. Y. Ahn (2014). “P42 Ebp1 regulates the proteasomal degradation of the p85 regulatory subunit of PI3K by recruiting a chaperone-E3 ligase complex HSP70/CHIP.” Cell Death Dis 5: e1131.
Kohlmeier, F., A. Maya-Mendoza and D. A. Jackson (2013). “EdU induces DNA damage response and cell death in mESC in culture.” Chromosome Res 21(1): 87-100.
Kuntz, A. L. and E. C. Oesterle (1998). “Transforming growth factor alpha with insulin stimulates cell proliferation in vivo in adult rat vestibular sensory epithelium.” J Comp Neurol 399(3): 413-423.
Kuo, B. R., E. M. Baldwin, W. S. Layman, M. M. Taketo and J. Zuo (2015). “In vivo Cochlear Hair Cell Generation and Survival by Coactivation of beta-Catenin and Atoh1.” J Neurosci 35(30): 10786-10798.
Lanford, P. J., Y. Lan, R. Jiang, C. Lindsell, G. Weinmaster, T. Gridley and M. W. Kelley (1999). “Notch signalling pathway mediates hair cell development in mammalian cochlea.” Nat Genet 21(3): 289-292.
Li, Y. M., Y. Pan, Y. Wei, X. Cheng, B. P. Zhou, M. Tan, X. Zhou, W. Xia, G. N. Hortobagyi, D. Yu and M. C. Hung (2004). “Upregulation of CXCR4 is essential for HER2-mediated tumor metastasis.” Cancer Cell 6(5): 459-469.
Lin, F. R., R. Thorpe, S. Gordon-Salant and L. Ferrucci (2011). “Hearing loss prevalence and risk factors among older adults in the United States.” J Gerontol A Biol Sci Med Sci 66(5): 582-590.
Liu, Z., B. J. Walters, T. Owen, M. A. Brimble, K. A. Steigelman, L. Zhang, M. M. Mellado Lagarde, M. B. Valentine, Y. Yu, B. C. Cox and J. Zuo (2012). “Regulation of p27Kip1 by Sox2 maintains quiescence of inner pillar cells in the murine auditory sensory epithelium.” J Neurosci 32(31): 10530-10540.
Lu, N., Y. Chen, Z. Wang, G. Chen, Q. Lin, Z. Y. Chen and H. Li (2013). “Sonic hedgehog initiates cochlear hair cell regeneration through downregulation of retinoblastoma protein.” Biochem Biophys Res Commun 430(2): 700-705.
Lumpkin, E. A., T. Collisson, P. Parab, A. Omer-Abdalla, H. Haeberle, P. Chen, A. Doetzlhofer, P. White, A. Groves, N. Segil and J. E. Johnson (2003). “Math1-driven GFP expression in the developing nervous system of transgenic mice.” Gene Expr Patterns 3(4): 389-395.
Madisen, L., T. A. Zwingman, S. M. Sunkin, S. W. Oh, H. A. Zariwala, H. Gu, L. L. Ng, R. D. Palmiter, M. J. Hawrylycz, A. R. Jones, E. S. Lein and H. Zeng (2010). “A robust and high-throughput Cre reporting and characterization system for the whole mouse brain.” Nat Neurosci 13(1): 133-140.
McGill, T. J. and H. F. Schuknecht (1976). “Human cochlear changes in noise induced hearing loss.” Laryngoscope 86(9): 1293-1302.
Mizutari, K., M. Fujioka, M. Hosoya, N. Bramhall, H. J. Okano, H. Okano and A. S. Edge (2013). “Notch inhibition induces cochlear hair cell regeneration and recovery of hearing after acoustic trauma.” Neuron 77(1): 58-69.
Montcouquiol, M. and J. T. Corwin (2001). “Intracellular signals that control cell proliferation in mammalian balance epithelia: key roles for phosphatidylinositol-3 kinase, mammalian target of rapamycin, and S6 kinases in preference to calcium, protein kinase C, and mitogen-activated protein kinase.” J Neurosci 21(2): 570-580.
Montgomery, S. C. and B. C. Cox (2016). “Whole Mount Dissection and Immunofluorescence of the Adult Mouse Cochlea.” J Vis Exp(107).
Neves, J., M. Uchikawa, A. Bigas and F. Giraldez (2012). “The prosensory function of Sox2 in the chicken inner ear relies on the direct regulation of Atoh1.” PLoS One 7(1): e30871.
Neves, J., I. Vachkov and F. Giraldez (2013). “Sox2 regulation of hair cell development: incoherence makes sense.” Hear Res 297: 20-29.
NIDCD. (2010, Jun. 16, 2010). “Quick Statistics on Hearing Loss.” 2010, from http://www.nidcd.nih.gov/health/statistics/Pages/quick.aspx.
Pan, W., Y. Jin, J. Chen, R. J. Rottier, K. P. Steel and A. E. Kiernan (2013). “Ectopic expression of activated notch or SOX2 reveals similar and unique roles in the development of the sensory cell progenitors in the mammalian inner ear.” J Neurosci 33(41): 16146-16157.
Puligilla, C. and M. W. Kelley (2017). “Dual role for Sox2 in specification of sensory competence and regulation of Atoh1 function.” Dev Neurobiol 77(1): 3-13.
Ryals, B. M. and E. W. Rubel (1988). “Hair cell regeneration after acoustic trauma in adult Coturnix quail.” Science 240(4860): 1774-1776.
Shen, W., M. S. Tremblay, V. A. Deshmukh, W. Wang, C. M. Filippi, G. Harb, Y. Q. Zhang, A. Kamireddy, J. E. Baaten, Q. Jin, T. Wu, J. G. Swoboda, C. Y. Cho, J. Li, B. A. Laffitte, P. McNamara, R.
Glynne, X. Wu, A. E. Herman and P. G. Schultz (2013). “Small-molecule inducer of beta cell proliferation identified by high-throughput screening.” J Am Chem Soc 135(5): 1669-1672.
Shi, F., Y. F. Cheng, X. L. Wang and A. S. Edge (2010). “Beta-catenin up-regulates Atoh1 expression in neural progenitor cells by interaction with an Atoh1 3′ enhancer.” J Biol Chem 285(1): 392-400.
Shi, F., L. Hu and A. S. Edge (2013). “Generation of hair cells in neonatal mice by beta-catenin overexpression in Lgr5-positive cochlear progenitors.” Proc Natl Acad Sci USA 110(34): 13851-13856.
Squatrito, M., M. Mancino, M. Donzelli, L. B. Areces and G. F. Draetta (2004). “EBP1 is a nucleolar growth-regulating protein that is part of pre-ribosomal ribonucleoprotein complexes.” Oncogene 23(25): 4454-4465.
Stankovic, K., C. Rio, A. Xia, M. Sugawara, J. C. Adams, M. C. Liberman and G. Corfas (2004). “Survival of adult spiral ganglion neurons requires erbB receptor signaling in the inner ear.” J Neurosci 24(40): 8651-8661.
Stern, D. F., M. P. Kamps and H. Cao (1988). “Oncogenic activation of p185neu stimulates tyrosine phosphorylation in vivo.” Mol Cell Biol 8(9): 3969-3973.
Suh, H., A. Consiglio, J. Ray, T. Sawai, K. A. D'Amour and F. H. Gage (2007). “In vivo fate analysis reveals the multipotent and self-renewal capacities of Sox2+ neural stem cells in the adult hippocampus.” Cell Stem Cell 1(5): 515-528.
Swoboda, J. G., J. Elliott, V. Deshmukh, L. de Lichtervelde, W. Shen, M. S. Tremblay, E. C. Peters, C. Y. Cho, B. Lu, S. Girman, S. Wang and P. G. Schultz (2013). “Small molecule mediated proliferation of primary retinal pigment epithelial cells.” ACS Chem Biol 8(7): 1407-1411.
Vermeer, P. D., L. A. Einwalter, T. O. Moninger, T. Rokhlina, J. A. Kern, J. Zabner and M. J. Welsh (2003). “Segregation of receptor and ligand regulates activation of epithelial growth factor receptor.” Nature 422(6929): 322-326.
Weiner, D. B., Y. Kokai, T. Wada, J. A. Cohen, W. V. Williams and M. I. Greene (1989). “Linkage of tyrosine kinase activity with transforming ability of the p185neu oncoprotein.” Oncogene 4(10): 1175-1183.
White, P. M., J. S. Stone, A. K. Groves and N. Segil (2012). “EGFR signaling is required for regenerative proliferation in the cochlea: conservation in birds and mammals.” Dev Biol 363(1): 191-200.
Xie, W., L. T. Chow, A. J. Paterson, E. Chin and J. E. Kudlow (1999). “Conditional expression of the ErbB2 oncogene elicits reversible hyperplasia in stratified epithelia and up-regulation of TGFalpha expression in transgenic mice.” Oncogene 18(24): 3593-3607.
Yamamoto, N., K. Tanigaki, M. Tsuji, D. Yabe, J. Ito and T. Honjo (2006). “Inhibition of Notch/RBP-J signaling induces hair cell formation in neonate mouse cochleas.” J Mol Med (Berl) 84(1): 37-45.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A method of expanding a population of inner ear cells, comprising contacting the cells with an effective amount of a Proliferation-Associated 2G4 (PA2G4) inhibitor.

2. The method of claim 1, wherein the PA2G4 inhibitor is WS3, WS6, or a derivative thereof, wherein WS3 is represented by the following structure: 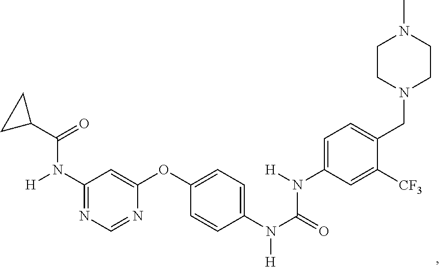 [SMILES: CN1CCN(Cc2ccc(NC(=O)Nc3ccc(Oc4cc(NC(=O)C5CC5)ncn4)cc3)cc2C(F)(F)F)CC1]
and WS6 is represented by the following structure:

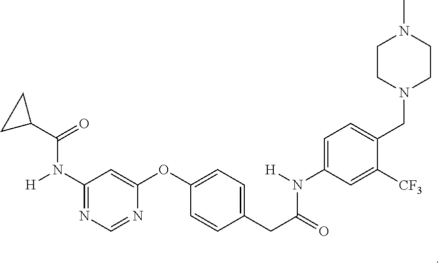 [SMILES: CN1CCN(Cc2ccc(NC(=O)Cc3ccc(Oc4cc(NC(=O)C5CC5)ncn4)cc3)cc2C(F)(F)F)CC1]

3. The method of claim 1, wherein the PA2G4 inhibitor is a siRNA molecule.

4. The method of claim 1, wherein the inner ear cells are Myo7⁺, Atoh1⁺ OCM⁺, Prestin⁺, or VGLUT3⁺.

5. The method of claim 1, wherein the inner ear cells are selected from the group consisting of inner hair cells, outer hair cells, vestibular hair cells, cochlear cells and vestibular supporting cells.

6. The method of claim 1, wherein the population of inner ear cells are in a cochlear tissue.

7. The method of claim 6, wherein the cochlear tissue is in vivo in a subject.

8. The method of claim 7, wherein the subject is a mammal.

9. The method of claim 8, wherein the mammal is a human.

10. The method of claim 6, wherein the cochlear tissue is in vitro.

11. A method of treating hearing loss in a subject in need thereof comprising applying to the inner ear or the organ of Corti of the subject an effective amount of an ERBB3 binding protein 1 (PA2G4) inhibitor.

12. The method of claim 11, wherein the inhibitor is administered into the scala tympani or the scala media.

13. The method of claim 11, wherein the PA2G4 inhibitor is in a sponge, a gel, a biopolymer, a tubing, or a pump.

14. The method of claim 13, wherein the PA2G4 inhibitor is a siRNA molecule.

15. The method of claim 13, wherein the PA2G4 inhibitor is WS3, WS6, or a derivative thereof.

16. The method of claim 15, wherein the PA2G4 inhibitor is injected at 0.005-60 ng/injection.

17. The method of claim 15, wherein the PA2G4 inhibitor is injected at 0.01-30 ng/injection.

18. A method of expanding a population of inner ear cells, comprising contacting the cells with an effective amount of an inhibitor of a negative ERBB3 regulator or a pharmaceutically acceptable salt of the inhibitor.

19. The method of claim 18, wherein the negative ERBB3 regulator is Proliferation-Associated 2G4 (PA2G4), Erbb2 interacting protein (ERBIN), ERBB receptor feedback inhibitor 1 (ERRFI1) and Protein Tyrosine Phosphatase, Receptor Type K (PTPRK).
